(19) United States
(12) Patent Application Publication
(10) Pub. No.: US 2024/0240169 A1
(43) Pub. Date: Jul. 18, 2024

(54) NOVEL SERINE PROTEASES

(71) Applicant: Bayer CropScience LP, St. Louis, MO (US)

(72) Inventors: Phillip GINGRICH, Houston, TX (US); Damian CURTIS, Davis, CA (US); Kevin BUGG, Lake Saint Louis, MO (US)

(21) Appl. No.: 18/245,378

(22) PCT Filed: Sep. 20, 2021
(86) PCT No.: PCT/US2021/051037

§ 371 Date: Mar. 15, 2023

Related U.S. Application Data
(60) Provisional application No. 63/081,271, filed on Sep. 21, 2020.

(51) Int. Cl. C12N 9/64; C12N 15/75

(52) U.S. Cl. CPC C12N 9/6424; C12N 15/75; C12Y 304/21; C12N 2800/101

(57) ABSTRACT

Serine proteases engineered to exhibit improvements, as well as cells and organisms comprising these novel serine proteases and methods of using the recombinant serine proteases to improve plant health or growth.

### CROSS-REFERENCE TO RELATED APPLICATIONS

The present application is a 35 U.S.C. § 371 national phase entry of International Application No. PCT/US2021/051037, filed on Sep. 20, 2021, which claims the benefit under 35 U.S.C. § 119 of U.S. Provisional Patent Application No. 63/081,271, filed Sep. 21, 2020, the contents of each of which are hereby incorporated by reference in their entirety.

### INCORPORATION OF SEQUENCE LISTING

The sequence listing contained in the file named “REPLACEMENT_BCS209004_ST25.txt”, which is 32 kilobytes as measured in Microsoft Windows operating system and was created on Sep. 20, 2021, is filed electronically herewith and incorporated herein by reference.

### TECHNICAL FIELD

The present invention relates to recombinant serine proteases engineered to exhibit improvements, as well as cells and organisms comprising these novel serine proteases. Methods of using the recombinant serine proteases to improve plant health or growth are further provided.

### BACKGROUND

Plant parasites and pathogens are major causes of yield loss in commercial crops, leading to significant economic costs. Treatment of plants with microbes via seed, foliar, or soil treatments can improve plant health by providing exogenous proteins to the plant or surrounding soil. Certain strains of bacteria protect plants from pests such as plant-parasitic nematodes and fungal plant pathogens due to expression of serine proteases. Serine proteases cleave peptide bonds at serine residues within a specific recognition site in a protein, and have been shown to degrade the intestinal tissue of nematodes as well as having antifungal activity specific to certain fungi. A continuing need exists in the art for the development of novel microbial compositions and methods that can be used to further improve crop plant growth and yield in a variety of agricultural field environments.

### SUMMARY

In one aspect, the present invention provides a recombinant DNA molecule comprising a nucleic acid sequence encoding a polypeptide with serine protease activity. The polypeptide may comprise an aspartic acid at a residue corresponding to residue 49 of SEQ ID NO: 1, or may comprise a histidine at a residue corresponding to residue 86 of SEQ ID NO: 1, or may comprise a serine at a residue corresponding to residue 244 of SEQ ID NO: 1, or may comprise a conservative substitution of any of these three residues, and comprises at least a first amino acid residue deletion relative to SEQ ID NO: 1. In certain embodiments, the polypeptide may comprise an aspartic acid at a residue corresponding to residue 49 of SEQ ID NO: 1, and a histidine at a residue corresponding to residue 86 of SEQ ID NO: 1, and a serine at a residue corresponding to residue 244 of SEQ ID NO: 1, or may comprise a conservative substitution of any or all of these three residues.

In some embodiments, polypeptides encoded by the recombinant DNA molecules provided may comprise an amino acid deletion of at least one residue corresponding to any of residues 177-243 of SEQ ID NO: 1, or an amino acid deletion of at least one residue corresponding to any of residues 181-240 of SEQ ID NO: 1. In one embodiment, the amino acid deletion corresponds to less than all of the residues of 181-240 of SEQ ID NO: 1. In further embodiments, the encoded polypeptide may comprise an amino acid deletion of at least the residues corresponding to residues 226-241 of SEQ ID NO: 1, or an amino acid deletion of at least the residues corresponding to residues 182-211 of SEQ ID NO: 1, or an amino acid deletion of at least the residues corresponding to residues 178-243 of SEQ ID NO: 1, or an amino acid deletion of at least the residues corresponding to residues 178-240 of SEQ ID NO: 1. The encoded polypeptide may have a sequence of any of SEQ ID NOs: 2 or 4-13.

Polypeptides encoded by the recombinant DNA molecules of the invention may exhibit increased serine protease activity compared to the polypeptide of SEQ ID NO: 1, or may exhibit increased nematicidal activity or increased antifungal activity compared to the polypeptide of SEQ ID NO: 1. The encoded polypeptides may also exhibit the same or increased substrate binding compared to the polypeptide of SEQ ID NO: 1. The encoded polypeptides may further comprise a glycine at a residue corresponding to residue 122 of SEQ ID NO: 1, a serine at a residue corresponding to residue 123 of SEQ ID NO: 1, a glycine at a residue corresponding to residue 124 of SEQ ID NO: 1, a glutamine at a residue corresponding to residue 125 of SEQ ID NO: 1, a tyrosine at a residue corresponding to residue 126 of SEQ ID NO: 1, a methionine at a residue corresponding to residue 146 of SEQ ID NO: 1, a serine at a residue corresponding to residue 147 of SEQ ID NO: 1, a leucine at a position corresponding to residue 148 of SEQ ID NO: 1, a glycine at a residue corresponding to residue 149 of SEQ ID NO: 1, a glycine at a residue corresponding to residue 150 of SEQ ID NO: 1, and a proline at a residue corresponding to residue 151 of SEQ ID NO: 1, or a conservative substitution of any of these residues.

In another aspect, the invention provides a polypeptide encoded by a recombinant DNA molecule provided herein. In certain embodiments, the polypeptide may comprise a sequence selected from SEQ ID NOs: 2 or 4-13, for example any of SEQ ID NOS: 2, 4-6 or 8-10.

In a further aspect, the invention provides a DNA construct comprising a recombinant DNA molecule provided herein operably linked to a promoter.

In yet another aspect, the invention provides a host cell comprising a recombinant DNA molecule provided herein. In some embodiments, the host cell may be a bacterial host cell, such as a bacterial host cell from the genus Bacillus, for example a Bacillus host cell selected from the group consisting of: Bacillus anthracis, Bacillus cereus, Bacillus thuringiensis, Bacillus mycoides, Bacillus pseudomycoides, Bacillus samanii, Bacillus gaemokensis, Bacillus weihenstephensis, and Bacillus toyoiensis. Formulations comprising the host cell and an agriculturally acceptable carrier are further provided. Plant seeds treated with the formulations disclosed herein are also provided.

In a further aspect, methods for stimulating plant growth and/or promoting plant health and/or controlling nematodes are provided, comprising applying the recombinant host cells provided herein to a plant growth medium, a plant, a plant seed, or an area surrounding a plant or a plant seed.

In another aspect, the invention provides transgenic plant cells comprising recombinant DNA molecules encoding the polypeptides provided herein, such as polypeptides comprising an aspartic acid at a residue corresponding to residue 49 of SEQ ID NO: 1, or a histidine at a residue corresponding to residue 86 of SEQ ID NO: 1, or a serine at a residue corresponding to residue 244 of SEQ ID NO: 1, or a conservative substitution of any of these three residues, and comprising at least a first amino acid residue deletion relative to SEQ ID NO: 1.

In yet another aspect, the invention provides transgenic plants, transgenic plant parts, and transgenic seeds comprising the recombinant DNA molecules encoding the polypeptides provided herein. The invention further provides transgenic plant cells, transgenic plants, transgenic plant parts, and transgenic seeds expressing or comprising the polypeptides disclosed herein.

In another aspect the invention provides transgenic plant cells, transgenic plants, transgenic plants parts, and transgenic seeds that comprise a recombinant DNA molecule encoding a polypeptide with serine protease activity, wherein said polypeptide comprises an aspartic acid at a residue corresponding to residue 49 of SEQ ID NO: 1, a histidine at a residue corresponding to residue 86 of SEQ ID NO: 1, and a serine at a residue corresponding to residue 244 of SEQ ID NO: 1, or a conservative substitution of any of these three residues. In another embodiment, the transgenic plant cells, transgenic plants, transgenic plants parts, and transgenic seeds express or comprise a polypeptide with serine protease activity, wherein said polypeptide comprises an aspartic acid at a residue corresponding to residue 49 of SEQ ID NO: 1, a histidine at a residue corresponding to residue 86 of SEQ ID NO: 1, and a serine at a residue corresponding to residue 244 of SEQ ID NO: 1, or a conservative substitution of any of these three residues. In one aspect of these embodiments, the polypeptide comprises SEQ ID NO: 1; in another the polypeptide comprises SEQ ID NO: 2; in another the polypeptide comprises any one of SEQ ID NOs: 4-13.

Plant cells, plants, plant parts, and seeds comprising the recombinant DNA molecules encoding the polypeptides provided herein may exhibit resistance to plant-parasitic nematodes or fungal resistance. Progeny plants comprising the recombinant DNA molecules disclosed herein are further provided.

Also provided herein are methods of producing a transgenic plant comprising transforming a plant cell with a recombinant DNA molecule of the invention to produce a transformed plant cell, and regenerating the transformed plant cell to produce a transgenic plant. The resulting transgenic plant may exhibit increased resistance to plant-parasitic nematodes or fungal resistance.

In still yet another aspect, the invention provides a method of producing a transgenic plant comprising the recombinant DNA molecules provided herein comprising crossing a transgenic plant comprising a recombinant DNA molecule of the invention with itself or another plant to produce one or more progeny plants, and selecting a progeny plant comprising the recombinant DNA molecule. The progeny plant may exhibit increased resistance to plant-parasitic nematodes or fungal resistance.

In certain aspects, the invention provides fusion proteins comprising: a) a targeting sequence, exosporium protein, or exosporium protein fragment that targets the fusion protein to the exosporium of a recombinant Bacillus host cell; and b) a polypeptide provided herein. In one embodiment, the targeting sequence, exosporium protein, or exosporium protein fragment comprises the sequence X₁-X₂-X₃-X₄-X₅-X₆-X₇-X₈-X₉-X₁₀-X₁₁-X₁₂-X₁₃-X₁₄-X₁₅-X₁₆, wherein:
X₁ is any amino acid or absent;
X₂ is phenylalanine (F), leucine (L), isoleucine (I), or methionine (M);
X₃ is any amino acid;
X₄ is proline (P) or serine (S);
X₅ is any amino acid;
X₆ is leucine (L), asparagine (N), serine (S), or isoleucine (I);
X₇ is valine (V) or isoleucine (I);
X₈ is glycine (G);
X₉ is proline (P);
X₁₀ is threonine (T) or proline (P);
X₁₁ is leucine (L) or phenylalanine (F);
X₁₂ is proline (P);
X₁₃ is any amino acid;
X₁₄ is any amino acid;
X₁₅ is proline (P), glutamine (Q), or threonine (T); and
X₁₆ is proline (P), threonine (T), or serine (S).

In another embodiment, the targeting sequence, exosporium protein, or exosporium protein fragment comprises SEQ ID NO: 14.

Recombinant host cells comprising the fusion protein are further provided, such as bacterial host cells. In certain embodiments, bacterial host cells from a Bacillus cereus family member comprising the fusion protein are provided. In one aspect of this embodiment, the Bacillus cereus family member is Bacillus anthracis, Bacillus cereus, Bacillus thuringiensis, Bacillus mycoides, Bacillus pseudomycoides, Bacillus samanii, Bacillus gaemokensis, Bacillus weihenstephanensis, or Bacillus toyonensis. In a particular aspect of this embodiment, the Bacillus cereus family member is Bacillus thuringiensis. Fermentation products comprising the bacterial host cells, and formulations comprising the fermentations products and an agriculturally acceptable carrier, are further provided.

In yet further aspects, methods for stimulating plant growth and/or promoting plant health and/or controlling nematodes are provided, comprising applying the formulations provided herein to a plant growth medium, a plant, a plant seed, or an area surrounding a plant or a plant seed.

### BRIEF DESCRIPTION OF THE DRAWINGS

FIG. 1 shows serine protease activity in whole broth cultures of Bacillus thuringiensis expressing no protein, expressing wild-type serine protease (SEQ ID NO: 1), or expressing a serine protease variant (SEQ ID NO: 2).

FIGS. 2a-2f shows a multiple sequence alignment of wild-type Bacillus firmus DS-1 intracellular serine protease (SEQ ID NO: 1), a variant of serine protease from Bacillus firmus (SEQ ID NO: 2), a homologous serine protease template for homology modeling (SEQ ID NO: 3), serine protease variants comprising intermediate deletions compared with SEQ ID NO: 2 (SEQ ID NOs: 4-6), and serine protease variants comprising extended deletions compared with SEQ ID NO: 2 (SEQ ID NOs: 7-13).

FIG. 3 shows (a) the structure of the serine protease template (SEQ ID NO: 3) used for homology modeling; and (b) the structure of wild-type Bacillus firmus DS-1 serine protease (SEQ ID NO: 1) following homology modeling.

FIG. 4 shows (a) the structure of the serine protease template (SEQ ID NO: 3) used for homology modeling; and (b) the structure of a variant of serine protease from Bacillus firmus strain DS-1 (Sep1 truncation) (SEQ ID NO: 2) following homology modeling.

FIG. 5 shows (a) the structure of the serine protease template (SEQ ID NO: 3) used for homology modeling; and (b) the structure of a variant of serine protease Intermediate_deletion_1 (SEQ ID NO: 4) following homology modeling.

FIG. 6 shows (a) the structure of the serine protease template (SEQ ID NO: 3) used for homology modeling; and (b) the structure of a variant of serine protease Intermediate_deletion_2 (SEQ ID NO: 5) following homology modeling.

FIG. 7 shows (a) the structure of the serine protease template (SEQ ID NO: 3) used for homology modeling; and (b) the structure of a variant of serine protease Intermediate_deletion_3 (SEQ ID NO: 6) following homology modeling.

FIG. 8 shows (a) the structure of the serine protease template (SEQ ID NO: 3) used for homology modeling; and (b) the structure of a variant of serine protease Extended_deletion_1 (SEQ ID NO: 7) following homology modeling.

FIG. 9 shows (a) the structure of the serine protease template (SEQ ID NO: 3) used for homology modeling; and (b) the structure of a variant of serine protease Extended_deletion_2 (SEQ ID NO: 8) following homology modeling.

FIG. 10 shows (a) the structure of the serine protease template (SEQ ID NO: 3) used for homology modeling; and (b) the structure of a variant of serine protease Extended_deletion_3 (SEQ ID NO: 9) following homology modeling.

FIG. 11 shows (a) the structure of the serine protease template (SEQ ID NO: 3) used for homology modeling; and (b) the structure of a variant of serine protease Extended_deletion_4 (SEQ ID NO: 10) following homology modeling.

FIG. 12 shows (a) the structure of the serine protease template (SEQ ID NO: 3) used for homology modeling; and (b) the structure of a variant of serine protease Extended_deletion_5 (SEQ ID NO: 11) following homology modeling.

FIG. 13 shows (a) the structure of the serine protease template (SEQ ID NO: 3) used for homology modeling; and (b) the structure of a variant of serine protease Extended_deletion_6 (SEQ ID NO: 12) following homology modeling.

FIG. 14 shows (a) the structure of the serine protease template (SEQ ID NO: 3) used for homology modeling; and (b) the structure of a variant of serine protease Extended_deletion_7 (SEQ ID NO: 13) following homology modeling.

### BRIEF DESCRIPTION OF THE SEQUENCES

SEQ ID NO: 1 is a wild-type Bacillus firmus DS-1 intracellular serine protease (UniProt Accession No. W7KRH1_BACFI).

SEQ ID NO: 2 is a variant of the Bacillus firmus DS-1 intracellular serine protease of SEQ ID NO: 1 in which residues corresponding to positions 181-240 of SEQ ID NO: 1 have been deleted.

SEQ ID NO: 3 is a serine protease from Bacillus pumilus (UniProt Accession No. P07518 (SUBT_BACPU)) used as a template for homology modeling.

SEQ ID NO: 4 is Intermediate_deletion_1, a variant serine protease in which residues corresponding to positions 226-242 of SEQ ID NO: 1 have been deleted.

SEQ ID NO: 5 is Intermediate_deletion_2, a variant serine protease in which residues corresponding to positions 212-241 of SEQ ID NO: 1 have been deleted.

SEQ ID NO: 6 is Intermediate_deletion_3, a variant serine protease in which residues corresponding to positions 182-211 of SEQ ID NO: 1 have been deleted, and a D has been substituted for T at the residue corresponding to position 243 of SEQ ID NO: 1.

SEQ ID NO: 7 is Extended_deletion_1, a variant serine protease in which residues corresponding to positions 181-243 of SEQ ID NO: 1 have been deleted.

SEQ ID NO: 8 is Extended_deletion_2, a variant serine protease in which residues corresponding to positions 178-240 of SEQ ID NO: 1 have been deleted.

SEQ ID NO: 9 is Extended_deletion_3, a variant serine protease in which residues corresponding to positions 178-243 of SEQ ID NO: 1 have been deleted.

SEQ ID NO: 10 is Extended_deletion_4, a variant serine protease in which residues corresponding to positions 177-243 of SEQ ID NO: 1 have been deleted.

SEQ ID NO: 11 is Extended_deletion_5, a variant serine protease in which residues corresponding to positions 176-243 of SEQ ID NO: 1 have been deleted.

SEQ ID NO: 12 is Extended_deletion_6, a variant serine protease in which residues corresponding to positions 175-243 of SEQ ID NO: 1 have been deleted.

SEQ ID NO: 13 is Extended_deletion_7, a variant serine protease in which residues corresponding to positions 174-243 of SEQ ID NO: 1 have been deleted.

SEQ ID NO: 14 is amino acids 1-41 of BclA from B. anthracis Sterne.

### DETAILED DESCRIPTION

Plant pests and pathogens can cause significant damage to crop plants, leading to substantial economic loss. Recent research has led to the development of bacterial strains expressing enzymes capable of reducing damage from plant pests or pathogens, which can be used as seed, foliar, or soil treatments to improve plant health and yield. However, improved enzymes for the treatment of certain pests responsible for particularly extensive yield loss, such as plant-parasitic nematodes, are needed.

Bacteria expressing serine proteases exhibit nematicidal as well as anti-fungal activity. The present invention provides serine proteases or serine protease variants with improved serine protease activity, which can be expressed in recombinant bacterial strains to improve plant health and increase yield.

### I. Serine Proteases

Serine proteases are one of the largest and mostly widely distributed classes of proteases. Serine proteases cleave peptide bonds at serine residues within a specific recognition site in a protein, and are frequently used by bacteria for nutrient scavenging in the environment. Serine proteases have also been shown to exhibit nematicidal activity through digestion of intestinal tissue in nematodes. Studies of Bacillus firmus strain DS-1, which shows nematicidal activity against Meloidogyne incognita and soybean cyst nematode, revealed that the serine protease produced by that strain has serine protease activity and degraded the intestinal tissues of nematodes. Geng. C., et al., Scientific Reports, 2016, vol. 6, no. 25012.

Other studies have shown that serine proteases have activity against pathogens such as fungal plant pathogens and oomycetes, such as Pythium. Dunne, et al., Microbiology, 2000, vol. 146, pp. 2069-2078, and Yen, Y., et al., Enzyme and Microbial Technology, 2006, vol. 39, pp. 311-317.

SEQ ID NOs: 1 and 2 provided herein are amino acid sequences for wild-type and variant enzymes that exhibit or are predicted to exhibit serine protease activity. Thus, for example, SEQ ID NO: 1 provides the amino acid sequence for a wild-type serine protease enzyme. SEQ ID NO: 2 provides the amino acid sequence for the same enzyme as in SEQ ID NO: 1, except for a deletion of amino acids 181-240 of SEQ ID NO: 1, such that SEQ ID NOs: 1 and 2 have 81% sequence similarity. The catalytic residues referenced in Geng, et al., 2016, are maintained in the variant serine protease amino acid sequence of SEQ ID NO: 2.

### II. Serine Proteases Variants

A polypeptide having serine protease activity as described herein may comprise a serine protease from Bacillus firmus, also referred to as a Bacillus firmus serine protease enzyme. In another embodiment, the serine protease from Bacillus firmus may be a Sep1 enzyme from a Bacillus firmus strain. In yet another embodiment, the serine protease may be a Sep1 enzyme from Bacillus firmus DS-1, which is SEQ ID NO: 1. In yet another embodiment, the serine protease can be a Sep1 enzyme from another Bacillus firmus strain.

Additionally, or alternatively, an enzyme provided herein having serine protease activity can comprise an amino acid sequence having at least one amino acid substitution or deletion relative to the sequence of a wild-type serine protease enzyme from a Bacillus firmus bacterium, wherein the amino acid substitution or deletion retains the catalytic residues of the wild-type enzyme and results in the same or increased serine protease activity as compared to the serine protease activity of the wild-type serine protease enzyme under the same conditions.

In some embodiments, the enzyme has increased serine protease activity as compared to the serine protease activity of the wild-type serine protease enzyme under the same conditions.

In some embodiments, a variant serine protease of the present invention decreases nematodes and/or nematode damage to a treated plant by at least about 0.5%, or by at least about 1%, or by at least about 2%, or by at least about 3%, or by at least about 5%, or by at least about 6%, or by at least about 7%, or by at least about 8%, or by at least about 9%, or by at least about 10%, or by at least about 11%, or by at least about 12% when compared to a plant produced under the same conditions but without treatment with the variant serine protease.

In some embodiments, a variant serine protease of the present invention decreases fungal growth and/or fungal damage to a treated plant by at least about 0.5%, or by at least about 1%, or by at least about 2%, or by at least about 3%, or by at least about 5%, or by at least about 6%, or by at least about 7%, or by at least about 8%, or by at least about 9%, or by at least about 10%, or by at least about 11%, or by at least about 12% when compared to a plant produced under the same conditions but without treatment with the variant serine protease.

Serine protease variants provided herein may comprise one or more mutations, deletions, or insertions relative to the base sequence from which they are derived. In certain embodiments, serine protease variants may have one or more mutations, deletions, or insertions compared with SEQ ID NO: 1. In other embodiments, serine protease variants may have one or more mutations, deletions, or insertions compared with any of SEQ ID NOs: 1-13. Serine protease variants derived from any of SEQ ID NOs: 1-13 may have the serine protease activity of any of SEQ ID NOs: 1-13. Serine protease variants derived from any of SEQ ID NOs: 1-13 may have increased serine protease activity compared with SEQ ID NOs: 1-13, or may have decreased serine protease activity compared with SEQ ID NOs: 1-13.

Serine protease variants provided herein may comprise one or more mutations, deletions, or insertions at a residue corresponding to a given residue of SEQ ID NO: 1. As used herein, “a residue corresponding to a given residue of SEQ ID NO: 1” means that, when the sequence comprising the residue is optimally aligned with SEQ ID NO: 1, the residue is aligned with the given residue of SEQ ID NO: 1. For example, if a sequence comprises an aspartic acid at a residue corresponding to residue 49 of SEQ ID NO: 1, that sequence comprises an aspartic acid aligned with residue 49 of SEQ ID NO: 1 when the two sequences are optimally aligned.

In certain embodiments, a serine protease variant provided herein comprises an amino acid deletion of at least one residue corresponding to any of residues 181-240 of SEQ ID NO: 1 (such as in SEQ ID NO: 2). In one aspect of this embodiment, the serine protease variant does not include deletion of all the residues 181-240 of SEQ ID NO: 1. In one embodiment, the amino acid deletion comprises at least two residues, at least three residues, at least four residues, at least five residues, at least six residues, at least seven residues, at least eight residues, at least nine residues, at least ten residues, at least 11 residues, at least 12 residues, at least 13 residues, at least 14 residues, at least 15 residues, at least 16 residues, at least about 20 residues, at least about 30 residues, at least about 40 residues, at least about 50 residues, at least about 51 residues, at least about 52 residues, at least about 53 residues, at least about 54 residues, at least about 55 residues, at least about 56 residues, at least about 57 residues, at least about 58 residues, or at least about 59 residues corresponding to any of residues 181-240 of SEQ ID NO: 1. In one aspect of this embodiment, the serine protease variant comprises an amino acid deletion of at least the residues corresponding to residue 182-211 of SEQ ID NO: 1 (SEQ ID NO: 6).

In other embodiments, the serine protease variant provided herein comprises an amino acid deletion of some or all of amino acid residues 181-240 of SEQ ID NO: 1 and of additional amino acid residues. Serine protease variants provided herein may further comprise an amino acid deletion of at least the residues corresponding to residue 226-242 of SEQ ID NO: 1 (SEQ ID NO: 4) or an amino acid deletion of at least the residues corresponding to residues 212-241 of SEQ ID NO: 1 (SEQ ID NO: 5). Serine protease variants provided herein may further comprise an amino acid deletion of at least the residues corresponding to residue 177-243 of SEQ ID NO: 1 (SEQ ID NO: 10) or an amino acid deletion of at least the residues corresponding to residue 178-243 of SEQ ID NO: 1 (SEQ ID NO: 9) or an amino acid deletion of at least the residues corresponding to residue 178-240 of SEQ ID NO: 1 (SEQ ID NO: 8) or an amino acid deletion of at least the residues corresponding to residues 181-243 of SEQ ID NO: 1 (SEQ ID NO: 7).

The serine protease variants provided may exhibit increased serine protease activity or increased antifungal activity compared to the polypeptide of SEQ ID NO: 1.

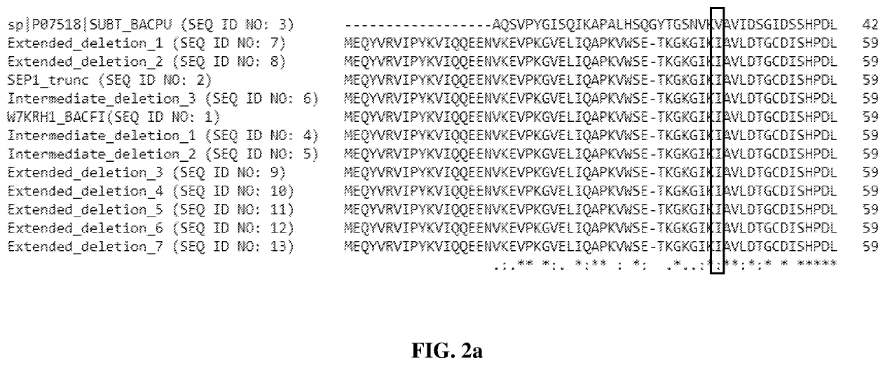

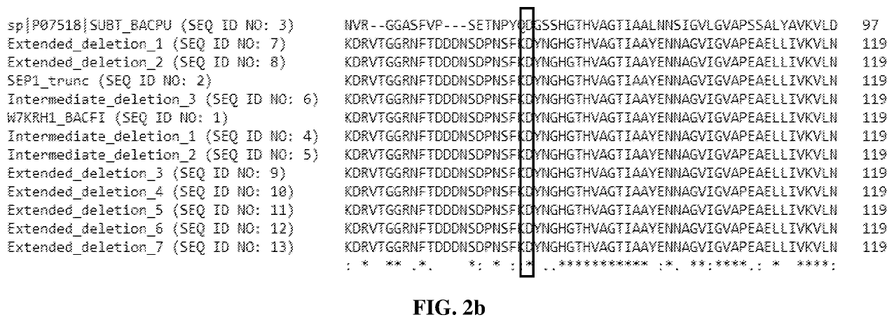

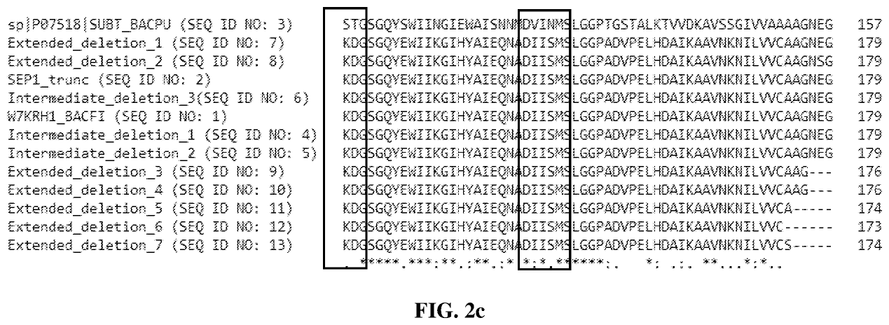

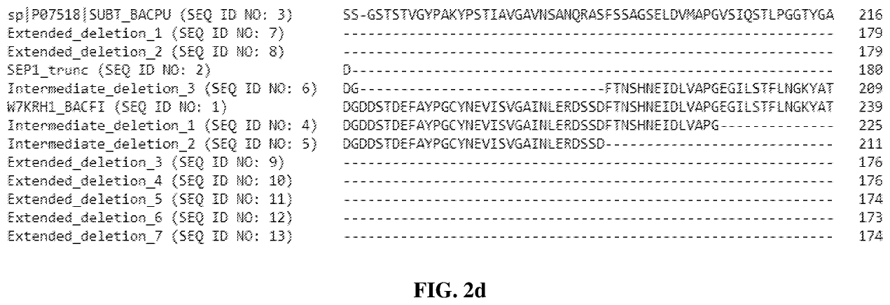

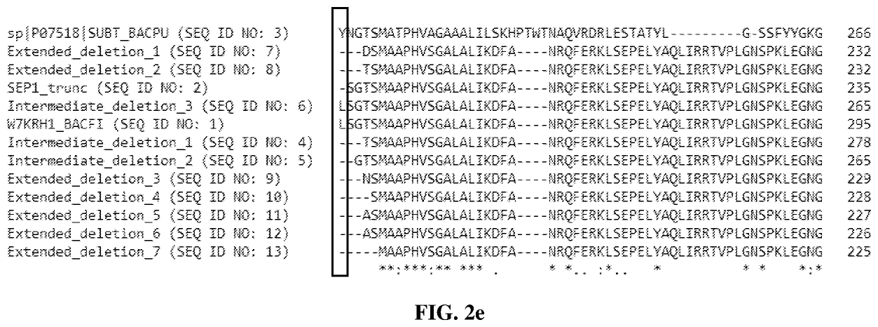

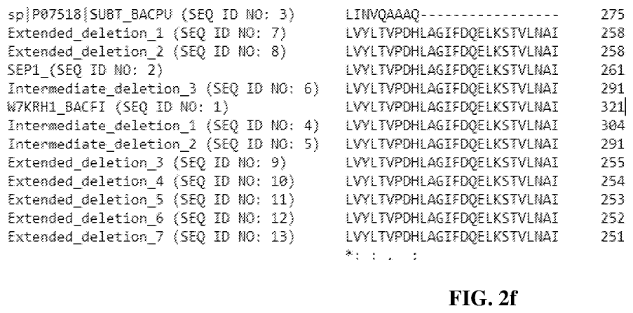

Serine protease variants disclosed herein may comprise one or more conservative mutations compared with a base sequence from which they are derived, for example any of SEQ ID NOs: 1-13, or in one illustrative embodiment, SEQ ID NO: 1. As shown in FIG. 2, serine protease variants share a significant degree of amino acid residue type similarity between aligned sequences. For example, amino acids may be classified according to the structure, size, electric charge, and influence on the solubility of amino acids in water into five groups: nonpolar aliphatic (glycine, alanine, valine, leucine, isoleucine, and proline), aromatic (phenylalanine, tyrosine, tryptophan), polar uncharged (serine, threonine, cysteine, methionine, asparagine, glutamine), negatively charged (aspartate, glutamate), and positively charged (lysine, arginine, histidine). Serine protease variants of the present invention may therefore comprise conservative mutations compared with any of SEQ ID NOs: 1-13 in which a nonpolar aliphatic residue is replaced with a different nonpolar aliphatic residue, an aromatic residue is replaced with a different aromatic residue, a polar uncharged residue is replaced with a different polar uncharged residue, a negatively charged residue is replaced with a different negatively charged residue, or a positively charged residue is replaced with a different positively charged residue. Serine protease variants of the present invention may also comprise conservative mutations compared with any of SEQ ID NOs: 1-13 in which an amino acid residue is replaced with a different amino acid residue having a similar R-group, for example serine/threonine, aspartate/glutamate, asparagine/glutamine, or leucine/isoleucine. Serine protease variants comprising conservative mutations may exhibit the same, greater, or less serine protease activity than the base sequence from which they are derived.

Serine protease variants provided herein may further comprise one or more conserved residues, regions, or domains associated with serine protease activity, or a conservative substitution thereof. For example, serine protease variants may comprise an aspartic acid residue at the position corresponding to residue 49 of SEQ ID NO: 1. Serine protease variants may comprise a histidine residue at the position corresponding to residue 86 of SEQ ID NO: 1. In addition or alternatively, serine protease variants may comprise a serine residue at the position corresponding to residue 244 of SEQ ID NO: 1. Asp49, His86, and Ser244 are involved in the catalytic triad in wild-type enzymes and are identified with rectangles in FIG. 2. Geng, et al., 2016. Serine protease variants may also comprise conservative mutations at any of the residues corresponding to the catalytic triad of Asp49, His86, and S244 in SEQ ID NO: 1. Serine protease variants may also comprise an asparagine residue or a conservative substitution thereof at the position corresponding to residue 177 of SEQ ID NO: 1.

Serine protease variants provided herein may further comprise a conserved substrate binding groove at the positions corresponding to Gly122, Ser123, Gly124, Gln125, and Tyr126 and/or Met146, Ser147, Leu148, Gly149, Gly150, Pro151 of SEQ ID NO: 1. In certain embodiments, serine protease variants provided herein comprise identical residues to SEQ ID NO: 1 at positions corresponding to Gly122, Ser123, Gly124, Gln125, and Tyr126 and/or Met146, Ser147, Leu148, Gly149, Gly150, Pro151 of SEQ ID NO: 1. Serine protease variants of the present invention may also comprise conservative mutations at the positions corresponding to residues 122-126 and 146-151 of SEQ ID NO: 1. The serine protease variants provided herein may exhibit the same or increased substrate binding compared with the polypeptide of SEQ ID NO: 1, and may comprise a glycine at a residue corresponding to residue 122 of SEQ ID NO: 1, a serine at a residue corresponding to residue 123 of SEQ ID NO: 1, a glycine at a residue corresponding to residue 124 of SEQ ID NO: 1, a glutamine at a residue corresponding to residue 125 of SEQ ID NO: 1, a tyrosine at a residue corresponding to residue 126 of SEQ ID NO: 1, a methionine at a residue corresponding to residue 146 of SEQ ID NO: 1, a serine at a residue corresponding to residue 147 of SEQ ID NO: 1, a leucine at a position corresponding to residue 148 of SEQ ID NO: 1, a glycine at a residue corresponding to residue 149 of SEQ ID NO: 1, a glycine at a residue corresponding to residue 150 of SEQ ID NO: 1, or a proline at a residue corresponding to residue 151 of SEQ ID NO: 1, or a conservative substitution of any of these residues.

Serine protease variants provided herein may also comprise non-natural amino acids substituted for the amino acids residues of SEQ ID NO: 1, so long as the serine protease variant having this substituted amino acids retains serine protease activity.

Serine protease variants may be synthetically produced or manipulated polypeptides, or may be produced through the fusion of two or more heterologous polypeptides. Methods of producing modified serine protease variants or DNA sequences encoding serine protease variants are well known in the art. Because of the degeneracy of the genetic code, a variety of different polynucleotide sequences can encode the serine proteases or serine protease variants disclosed herein. All possible triplet codons (and where U also replaces T) and the amino acid encoded by each codon is well-known in the art. In addition, it is well within the capability of one of skill in the art to create alternative polynucleotide sequences encoding the same, or essentially the same, mutant polypeptides of the subject disclosure. Allelic variants of the nucleotide sequences encoding a wild-type or mutant polypeptide of the present disclosure are also encompassed within the scope of the disclosure.

The invention further provides recombinant DNA molecules encoding the serine proteases or serine protease variants disclosed herein. Further provided are recombinant DNA constructs comprising a DNA molecule encoding a serine protease or serine protease variant of the invention, operably linked with a promoter or other regulatory element. In certain embodiments, the promoter may be heterologous with respect to the recombinant DNA molecule. As used herein, the term “heterologous” refers to the combination of two or more DNA molecules when such a combination is not normally found in nature. For example, the two DNA molecules may be derived from different species and/or the two DNA molecules may be derived from different genes, e.g., different genes from the same species or the same genes from different species. A regulatory element is thus heterologous with respect to an operably linked transcribable DNA molecule if such a combination is not normally found in nature, i.e., the recombinant DNA molecule does not naturally occur operably linked to the promoter.

As used herein, a “recombinant polypeptide” is a polypeptide comprising a combination of polypeptides that would not naturally occur together without human intervention. For instance, a recombinant polypeptide may be a polypeptide that is comprised of at least two polypeptides heterologous with respect to each other, a polypeptide that comprises a polypeptide sequence that deviates from polypeptide sequences that exist in nature, a polypeptide that comprises a synthetic polypeptide sequence or a polypeptide expressed by a recombinant DNA sequence that has been incorporated into a host cell's DNA by genetic transformation or gene editing.

Reference in this application to an “isolated polypeptide”, or an equivalent term or phrase, is intended to mean that the polypeptide is one that is present alone or in combination with other compositions, but not within its natural environment. Similarly, a DNA molecule encoding a serine protease or any naturally occurring serine protease variant would be an isolated DNA molecule so long as the nucleotide sequence was not within the DNA of the bacterium from which the sequence encoding the protein is naturally found. A synthetic nucleotide sequence encoding the amino acid sequence of the naturally occurring serine protease would be considered to be isolated for the purposes of this disclosure. For the purposes of this disclosure, any transgenic nucleotide sequence, i.e., the nucleotide sequence of the DNA inserted into the genome of the cells of a plant or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

This disclosure further contemplates that improved variants of serine proteases can be engineered within a cell by using various gene editing methods known in the art. Such technologies used for genome editing include, but are not limited to, ZFN (zinc-finger nuclease), meganucleases, TALEN (Transcription activator-like effector nucleases), and CRISPR (Clustered Regularly Interspaced Short Palindromic Repeats)/Cas (CRISPR-associated) systems. These genome editing methods can be used to alter the serine protease coding sequence transformed within a plant cell to a different serine protease coding sequence. Specifically, through these methods, one or more codons within the serine protease coding sequence is altered to engineer a new protein amino acid sequence. Alternatively, a fragment within the coding sequence is replaced or deleted, or additional DNA fragments are inserted into the coding sequence, to engineer a new serine protease coding sequence. The new coding sequence can encode a serine protease with new properties such as increased activity or spectrum against insect pests, as well as provide activity against an insect pest species wherein resistance has developed against the original insect toxin protein. The plant cell comprising the gene edited serine protease coding sequence can be used by methods known in the art to generate cells, including bacterial or plant cells, expressing the modified serine protease.

For serine protease enzymes described herein, “sequence identity” or “percent sequence identity” or “% sequence identity” is determined by aligning the entire length of the sequences in such a way as to obtain optimal matching so that the minimal number of edit operations (e.g., inserts, deletions and substitutions) are needed in order to transform the one sequence into an exact copy of the other sequence being aligned. The EMBOSS Needle Pairwise Sequence Alignment, which is an algorithm that is available through the European Bioinformatics Institute (EMBL-EBI) website, is one example of such analysis.

Alternatively or in addition, the enzyme having serine protease activity can comprise an amino acid sequence defined as having at least 70% identity to one or more of SEQ ID NOs: 1-13.

For example, the enzyme having serine protease activity can comprise an amino acid sequence defined as having at least 75% identity to one or more of SEQ ID NOs: 1-13.

The enzyme having serine protease activity can comprise an amino acid sequence defined as having at least 80% identity to one or more of SEQ ID NOs: 1-13.

The enzyme having serine protease activity can comprise an amino acid sequence defined as having at least 85% identity to one or more of SEQ ID NOs: 1-13.

The enzyme having serine protease activity can comprise an amino acid sequence defined as having at least 90% identity to one or more of SEQ ID NOs: 1-13.

The enzyme having serine protease activity can comprise an amino acid sequence defined as having at least 95% identity to one or more of SEQ ID NOs: 1-13.

The enzyme having serine protease activity can comprise an amino acid sequence defined as having at least 98% identity to one or more of SEQ ID NOs: 1-13.

The enzyme having serine protease activity can comprise an amino acid sequence defined as having at least 99% identity to one or more of SEQ ID NOs: 1-13.

The enzyme having serine protease activity can comprise an amino acid sequence having 100% identity to any of SEQ ID NOs: 1-13.

For example, the enzyme can comprise any of SEQ ID NOs: 1-13.

Alternatively, the enzyme can consist of any of SEQ ID NOs: 1-13.

In addition, the enzyme having serine protease activity and having 70%, 75%, 80%, 85%, 90%, 95%, 98%, or 99% sequence identity to any one or more of SEQ ID NOs: 1-13 may comprise the deletion in SEQ ID NO: 2 (of amino acids 181-240 of SEQ ID NO: 1).

### III. Free Enzymes

Any of the enzymes described herein can also be used as free enzymes or as enzymes expressed in recombinant microorganisms.

### IV. Expression of Serine Protease Variants on the Exosporium of Bacillus cereus

Serine proteases and serine protease variants of the invention may also be expressed as fusion proteins comprising a targeting sequence, exosporium protein, or exosporium protein fragment that targets the fusion protein to the exosporium of a recombinant Bacillus cereus family member. The fusion proteins further comprise a polypeptide having serine protease activity as described herein. When expressed in a Bacillus cereus bacterium, these fusion proteins are targeted to the exosporium layer of the spore and are physically oriented such that the serine protease is displayed on the outside of the spore.

A fusion protein comprising a serine protease or serine protease variant described herein may comprise any targeting sequence capable of targeting the fusion protein to the exosporium of a recombinant Bacillus cereus family member. It was previously discovered that certain sequences from the N-terminal regions of BclA and BclB could be used to target a peptide or protein to the exosporium of a Bacillus cereus family member endospore (see U.S. Patent Application Publication Nos. 2010/0233124 and 2011/0281316, and Thompson et al., “Targeting of the BclA and BclB Proteins to the Bacillus anthracis Spore Surface”, Molecular Microbiology 70(2):421-34 (2008)). It was also found that the BetA/BAS3290 protein of Bacillus anthracis localized to the exosporium. Further targeting sequences, as well as exosporium proteins and fragments of exosporium proteins, which can be incorporated into a fusion protein and used to target a peptide or protein of interest to the exosporium of a recombinant Bacillus cereus family member are described in U.S. Patent Application Publication Nos. 2016/0031948 and 2016/0108096, which are incorporated by reference herein in their entirety.

Bacillus is a genus of rod-shaped bacteria. The Bacillus cereus family of bacteria includes any Bacillus species that is capable of producing an exosporium. Thus, the Bacillus cereus family of bacteria includes the species Bacillus anthracis, Bacillus cereus, Bacillus thuringiensis, Bacillus mycoides, Bacillus pseudomycoides, Bacillus samanii, Bacillus gaemokensis, Bacillus weihenstephanensis, and Bacillus toyonensis. Under stressful environmental conditions, Bacillus cereus family bacteria undergo sporulation and form oval endospores that can stay dormant for extended periods of time. The outermost layer of the endospores is known as the exosporium and comprises a basal layer surrounded by an external nap of hair-like projections. Filaments on the hair-like nap are predominantly formed by the collagen-like glycoprotein BclA, while the basal layer is comprised of a number of different proteins. Another collagen-related protein, BclB, is also present in the exosporium and exposed on endospores of Bacillus cereus family members. BclA, the major constituent of the surface nap, has been shown to be attached to the exosporium with its amino-terminus (N-terminus) positioned at the basal layer and its carboxy-terminus (C-terminus) extending outward from the spore.

The scientific literature describes the Bacillus cereus “family” or “group” as a subgroup within the genus Bacillus. See Priest et al., “Population Structure and Evolution of the Bacillus cereus Group,” J. Bacteriology, 2004, vol. 186, no. 23, pp. 7959-7970; Peng et al., “The Regulation of Exosporium-Related Genes in Bacillus thuringiensis,” Nature Scientific Reports, 2016, vol. 6, no. 19005, pp. 1-12. Peng et al. states:

Spores of the B. cereus group are complex, multilayered structures. The nucleoid containing core is enclosed within a peptidoglycan cortex, which is surrounded by the spore coat. Spores of all the B. cereus group species are encircled by an additional loose-fitting layer called the exosporium, which is not present on other species such as Bacillus subtilis, for which the coat constitutes the outermost layer of the mature spore. The exosporium is a balloon-like layer that acts as the outer permeability barrier of the spore and contributes to spore survival and virulence.

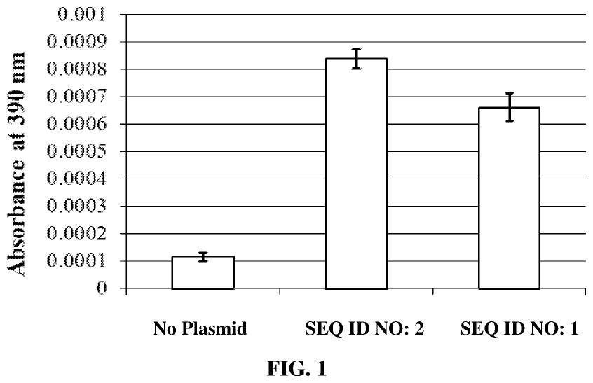

The targeting sequence, exosporium protein or exosporium protein fragment of the present invention may also be described in terms of a motif that provides the targeting function. A sequence alignment of the amino-terminal region of BclA (SEQ ID NO: 14) with the corresponding amino-terminal regions of a number of other Bacillus cereus family member exosporium proteins shows that there is a conserved motif at amino acids 20-35 of BclA, with a more highly conserved motif at amino acids 25-35 of BclA. For more detail, see the alignment provided in FIGS. 1A and 1B of International Publication No. WO 2019/060574. This more highly conserved region is the recognition sequence for ExsFA/BxpB/ExsFB and homologs, which direct and assemble the described exosporium proteins on the surface of the exosporium.

Furthermore, while amino acids 20-35 of BclA are conserved, and amino acids 25-35 are more conserved, some degree of variation can occur in this region without affecting the ability of the targeting sequence to target a protein to the exosporium. Sequences having a targeting sequence identity as low as 43.8% with amino acids 20-35 of BclA (SEQ ID NO: 14), wherein the identity with amino acids 25-35 of BclA is 54.5%, retain the ability to target fusion proteins to the exosporium. Some data are provided in Table 58 in Example 59 of PCT Publication No. WO 2016/044661, which is incorporated herein by reference in its entirety.

These data show that targeting of a protein of interest (e.g., an enzyme) to the exosporium proteins can be achieved using targeting sequences having 50-68.8% identity to amino acids 20-35 of BclA (SEQ ID NO: 14), wherein the identity to amino acids 25-35 of BclA is 63.6% to 81.8%. Such motif is present in a targeting sequence, exosporium protein, or exosporium protein fragment that targets the fusion protein to the exosporium of the recombinant Bacillus bacterium and comprises the sequence X₁-X₂-X₃-X₄-X₅-X₆-X₇-X₈-X₉-X₁₀-X₁₁-X₁₂-X₁₃-X₁₄-X₁₅-X₁₆, wherein:
X₁ is any amino acid or absent;
X₂ is phenylalanine (F), leucine (L), isoleucine (I), or methionine (M);
X₃ is any amino acid;
X₄ is proline (P) or serine (S);
X₅ is any amino acid;
X₆ is leucine (L), asparagine (N), serine (S), or isoleucine (I);
X₇ is valine (V) or isoleucine (I);
X₈ is glycine (G);
X₉ is proline (P);
X₁₀ is threonine (T) or proline (P);
X₁₁ is leucine (L) or phenylalanine (F);
X₁₂ is proline (P);
X₁₃ is any amino acid;
X₁₄ is any amino acid;
X₁₅ is proline (P), glutamine (Q), or threonine (T); and
X₁₆ is proline (P), threonine (T), or serine (S).

Any of the targeting sequences, exosporium proteins, or exosporium protein fragments can be used to target any protein or peptide of interest, including the proteins having serine protease activity described herein, to the exosporium of a recombinant Bacillus cereus family member.

During sporulation of a recombinant Bacillus cereus family member expressing any of the fusion proteins described herein, the targeting motif, exosporium protein, or exosporium protein fragment is recognized by the spore exosporium assembly machinery and directed to the exosporium, resulting in display of the protein or peptide of interest portion of the fusion protein (e.g., the enzyme having serine protease activity) on the outside of the spore.

The use of different targeting sequences allows for control of the expression level of the fusion protein on the surface of the Bacillus cereus family member spore. Use of certain of the targeting sequences described herein will result in a higher level of expression of the fusion protein, whereas use of others of the targeting sequences will result in lower levels of expression of the fusion protein on the surface of the spore.

In any of the fusion proteins described herein, the targeting sequence, exosporium protein, or exosporium protein fragment can comprise the amino acid sequence GXT at its carboxy terminus, wherein X is any amino acid.

In any of the fusion proteins described herein, the targeting sequence, exosporium protein, or exosporium protein fragment, can comprise an alanine residue at the position of the targeting sequence that corresponds to amino acid 20 of SEQ ID NO: 14.

In any of the fusion proteins described herein, the targeting sequence, exosporium protein, or exosporium protein fragment can further comprise a methionine, serine, or threonine residue at the amino acid position immediately preceding the first amino acid of the targeting sequence, exosporium protein, or exosporium protein fragment or at the position of the targeting sequence that corresponds to amino acid 20 of SEQ ID NO: 14.

This Bacillus exosporium display (BEMD) system can be used to deliver a serine protease or serine protease variant as described herein to plants (e.g., to plant foliage, fruits, flowers, stems, or roots) or to a plant growth medium such as soil. Enzymes and proteins delivered to the soil or another plant growth medium in this manner persist and exhibit activity in the soil for extended periods of time. Introduction of recombinant Bacillus cereus family member bacteria expressing fusion proteins comprising the serine proteases or serine protease variants described herein into soil or the rhizosphere of a plant leads to a beneficial enhancement of plant growth and/or to control pests, such as nematodes, in many different soil conditions. The use of the BEMD to create these enzymes allows them to continue to exert their beneficial results to the plant and the rhizosphere over the first months of a plant's life.

In addition, the BEMD system can be modified such that the exosporium of the recombinant Bacillus cereus family member can be removed from the spore, generating exosporium fragments containing the fusion proteins, as described in International Publication No. WO 2016/044661). The exosporium fragments can also be used to deliver the serine proteases or serine protease variants to plants in a cell-free preparation.

### V. Formulations

Formulations are further provided comprising a host cell comprising any of the recombinant serine proteases provided herein. In certain examples, the host cell may be a bacterial host cell, for example a recombinant Bacillus cereus family member. A formulation provided herein may further comprise an agriculturally acceptable carrier.

In another embodiment, a formulation provided herein may comprise exosporium fragments derived from a host cell comprising any of the recombinant serine proteases provided herein. In some examples, the exosporium fragments may be derived from a bacterial host cell, such as recombinant Bacillus cereus family member. The formulation may further comprise an agriculturally acceptable carrier.

### VI. Transgenic Plants

An aspect of the invention includes transgenic plant cells, transgenic plant tissues, transgenic plants, and transgenic seeds that comprise the recombinant DNA molecules encoding serine proteases and serine protease variants provided by the invention. The invention further provides transgenic plants cells, transgenic plant tissues, transgenic plants, and transgenic seeds expressing or comprising the serine proteases or serine protease variants disclosed herein. These plant cells, plant tissues, plants, and seeds comprising the recombinant DNA molecules, the serine proteases, or the serine protease variants may exhibit resistance to plant-parasitic nematodes or fungal resistance.

Suitable methods for transformation of host plant cells for use with the current invention include virtually any method by which DNA can be introduced into a cell (for example, where a recombinant DNA construct is stably integrated into a plant chromosome) and are well known in the art. An exemplary and widely utilized method for introducing a recombinant DNA construct into plants is the Agrobacterium transformation system, which is well known to those of skill in the art. Transgenic plants can be regenerated from a transformed plant cell by the methods of plant cell culture.

The transgenic plants, progeny, seeds, plant cells, and plant parts of the invention may also contain one or more additional transgenic traits. Additional transgenic traits may be introduced by crossing a plant containing a transgene comprising the recombinant DNA molecules provided by the invention with another plant containing an additional transgenic trait(s). As used herein, “crossing” means breeding two individual plants to produce a progeny plant. Two transgenic plants may thus be crossed to produce progeny that contain the transgenic traits. As used herein “progeny” means the offspring of any generation of a parent plant, and transgenic progeny comprise a DNA construct provided by the invention and inherited from at least one parent plant. Alternatively, additional transgenic trait(s) may be introduced by co-transforming a DNA construct for that additional transgenic trait(s) with a DNA construct comprising the recombinant DNA molecules provided by the invention (for example, with all the DNA constructs present as part of the same vector used for plant transformation) or by inserting the additional trait(s) into a transgenic plant comprising a DNA construct provided by the invention or vice versa (for example, by using any of the methods of plant transformation on a transgenic plant or plant cell).

Transgenic plants and progeny that contain a transgenic trait provided by the invention may be used with any breeding methods that are commonly known in the art. In plant lines comprising two or more transgenic traits, the transgenic traits may be independently segregating, linked, or a combination of both in plant lines comprising three or more transgenic traits. Back-crossing to a parental plant and out-crossing with a non-transgenic plant are also contemplated, as is vegetative propagation. Descriptions of breeding methods that are commonly used for different traits and crops are well known to those of skill in the art. To confirm the presence of the transgene(s) in a particular plant or seed, a variety of assays may be performed. Such assays include, for example, molecular biology assays, such as Southern and northern blotting, PCR, and DNA sequencing; biochemical assays, such as detecting the presence of a protein product, for example, by immunological means (ELISAs and Western blots) or by enzymatic function; plant part assays, such as leaf or root assays; and also, by analyzing the phenotype of the whole plant.

### VII. Treated Seeds

Treated plant seeds are further provided. The plant seed may be treated with a host cell comprising any of the recombinant serine proteases provided herein, such as a recombinant bacterium. The recombinant bacterium may be a Bacillus cereus family member. The recombinant bacterium may express any of the serine proteases or serine protease variants described herein.

Treated plant seeds are further provided which may be treated with any of the exosporium fragments described herein. The exosporium fragments can be derived from any of the Bacillus cereus family members described herein. The exosporium fragments can comprise any of the serine proteases or serine protease variants described herein.

Plant seeds treated with any of the formulations described herein are further provided.

In any of the treated plant seeds provided, the plant seed may be coated with a host cell comprising any of the recombinant serine proteases provided herein, such as a recombinant bacterium, or with exosporium fragments comprising a recombinant serine protease provided, or with a formulation comprising a recombinant serine protease provided.

The host cells, recombinant bacteria, exosporium fragments, or formulations may be used as seed treatments, e.g., seed coatings or dressings. Seed coating or dressing formulations may be in the form of a liquid carrier formulation, a slurry formulation, or a powder formulation.

Seed coating or dressing formulations may be applied with conventional additives that are provided to make the seed treatment have sticky qualities to stick to and coat the seeds. Suitable additives comprise: talcs, graphites, gums, stabilizing polymers, coating polymers, finishing polymers, slip agents for seed flow and plantability, cosmetic agents, and cellulosic materials such as carboxymethyl cellulose and the like.

The seed treatment formulations may further comprise colorant agents and/or other additives.

The seed treatment formulations(s) may be applied to seeds in a suitable carrier such as water or a powder. The seeds can then be allowed to dry and planted in conventional fashion. The host cells, recombinant bacteria, exosporium fragments, or formulations can be applied directly to the seed as a solution or in combination with other commercially available additives. For example, the recombinant host cell, recombinant bacterium, exosporium fragment, or formulation can be applied in combination with seedling-acceptable carrier(s) (e.g., a liquid carrier or a solid carrier).

Solutions containing the recombinant host cell, recombinant bacterium, exosporium fragment, or formulation can be sprayed or otherwise applied to the seed (e.g., in a seed slurry or a seed soak).

Solid or dry materials containing recombinant host cells, recombinant bacteria, exosporium fragments, or formulations are also useful to promote effective seedling germination, growth, and protection during early seedling establishment.

The recombinant host cell, recombinant bacterium, exosporium fragment, or formulation can be used with a solubilizing carrier such as water, a buffer (e.g., citrate or phosphate buffer), other treating agents (e.g., alcohol or another solvent), and/or any soluble agent.

In addition, small amounts of drying agent enhancers, such as lower alcohols, etc. can be used in seed coating formulations.

Surfactants, emulsifiers and preservatives can also be added at relatively low (e.g., about 0.5% w/v or less) levels in order to enhance the stability of the seed coating product.

Seeds can be treated using a variety of methods including, but not limited to, pouring, pumping, drizzling, or spraying an aqueous solution containing the recombinant the recombinant host cell, recombinant bacterium, exosporium fragment, or formulation on or over a seed; or spraying or applying the recombinant host cell, recombinant bacterium, exosporium fragment, or formulation onto a layer of seeds either with or without the use of a conveyor system.

Mixing devices useful for seed treatment include but are not limited to tumblers, mixing basins, mixing drums, and fluid application devices that include basins or drums used to contain the seed while coating.

After seed treatment, the seed may be air-dried or a stream of dry air may be optionally used to aid in the drying of the seed coatings.

Seed treatments containing the recombinant host cell, recombinant bacterium, exosporium fragment, or formulation can be applied using any commercially available seed treatment machinery or can also be applied using any acceptable non-commercial method(s) such as the use of syringes or any other seed treatment device.

### VIII. Methods for Stimulating Plant Growth and/or Promoting Plant Health and/or Controlling Plant Pathogens

Methods for stimulating plant growth and/or promoting plant health and/or controlling plant pests, such as nematodes, and/or controlling plant pathogens are provided. The methods comprise applying a host cell comprising a serine protease provided herein to a plant growth medium, a plant, a plant seed, or an area surrounding a plant or a plant seed and contacting the plant pest with a recombinant host cell. The recombinant host cell can comprise any of the recombinant host cells described herein. The recombinant host cell can express any of the serine proteases described herein.

Another method for stimulating plant growth and/or promoting plant health and/or controlling plant pests, such as nematodes, and/or controlling plant pathogens is provided. The method comprises applying exosporium fragments to a plant growth medium, a plant, a plant seed, or an area surrounding a plant or a plant seed or contacting the plant pest with exosporium fragments. The exosporium fragments can comprise exosporium fragments derived from any of the recombinant Bacillus cereus family members described herein. The exosporium fragments can comprise any of the serine proteases described herein.

Yet another method for stimulating plant growth and/or promoting plant health and/or controlling plant pests, such as nematodes, and/or controlling plant pathogens is provided. The method comprises applying a formulation provided herein to a plant growth medium, a plant, a plant seed, or an area surrounding a plant or a plant seed. The formulation can comprise any of the formulations described herein.

In any of the methods described herein, the method can further comprise inactivating the recombinant bacterial host cell prior to applying the recombinant bacterial host cell to the plant growth medium, the plant, the plant seed, or the area surrounding the plant or the plant seed.

In any of the methods described herein, the method can comprise applying the recombinant host cell, recombinant bacterium, exosporium fragment, or formulation to the plant growth medium.

In any of the methods described herein involving the use of a plant growth medium, the plant growth medium can comprise soil, water, an aqueous solution, sand, gravel, a polysaccharide, mulch, compost, peat moss, straw, logs, clay, soybean meal, yeast extract, or a combination thereof.

The plant growth medium can comprise a fertilizer.

Any of the methods described herein can further comprise supplementing the plant growth medium with a substrate for an enzyme. Suitable substrates include, but are not limited to protein meal, casein, gelatin, albumin, or a combination of any thereof.

In any of the methods described herein, the method can comprise applying the recombinant host cell, recombinant bacterium, exosporium fragment, or formulation to the plant.

For example, the method can comprise applying the recombinant host cell, recombinant bacterium, exosporium fragment, or formulation to roots of the plant.

Alternatively or in addition, the method can comprise applying the recombinant host cell, recombinant bacterium, exosporium fragment, or formulation foliarly.

In any of the methods described herein, the method can comprise applying the recombinant host cell, recombinant bacterium, exosporium fragment, or formulation to the plant seed.

Where the method comprises applying the recombinant host cell, recombinant bacterium, exosporium fragment, or formulation to the plant seed, applying the recombinant host cell, recombinant bacterium, exosporium fragment, or formulation to the plant seed can comprise: (a) applying recombinant host cells, recombinant bacteria, exosporium fragments, or formulations to the plant seed at the time of planting; or (b) coating the plant seed with the recombinant host cells, recombinant bacteria, exosporium fragments, or formulations.

In any of the methods described herein, the plant pests that are controlled can be phytoparasitic pests from the phylum Nematoda, for example, Aglenchus spp., Anguina spp., Aphelenchoides spp., Belonolaimus spp., Bursaphelenchus spp., Cacopaurus spp., Criconemella spp., Criconemoides spp., Ditylenchus spp., Dolichodorus spp., Globodera spp., Helicotylenchus spp., Hemicriconemoides spp., Hemicycliophora spp., Heterodera spp., Hoplolaimus spp., Longidorus spp., Lygus spp., Meloidogyne spp., Meloinema spp., Nacobbus spp., Neotylenchus spp., Paralongidorus spp., Paraphelenchus spp., Paratrichodorus spp., Pratylenchus spp., Pseudohalenchus spp., Psilenchus spp., Punctodera spp., Quinisulcius spp., Radopholus spp., Rotylenchulus spp., Rotylenchus spp., Scutellonema spp., Subanguina spp., Trichodorus spp., Tylenchulus spp., Tylenchorhynchus spp., Xiphinema spp.

In any of the methods described herein, plants grown in the presence of the recombinant host cell, recombinant bacterium, exosporium fragment, or formulation can exhibit increased growth as compared to plants grown in the absence of the enzyme or the microorganism under the same conditions.

In any of the methods described herein, seeds to which the recombinant host cell, recombinant bacterium, exosporium fragment, or formulation have been applied can exhibit increased germination rates as compared to seeds to which the enzyme or microorganism has not been applied, under the same conditions.

In any of the methods described herein, plants grown in the presence of the recombinant host cell, recombinant bacterium, exosporium fragment, or formulation can exhibit increased nutrient uptake as compared to plants grown in the absence of the enzyme or the microorganism, under the same conditions.

In any of the methods described herein, plants grown in the presence of the recombinant host cell, recombinant bacterium, exosporium fragment, or formulation can exhibit decreased susceptibility to a pest, such as nematodes, as compared to plants grown in the absence of the enzyme or the microorganism, under the same conditions.

In any of the methods described herein, plants grown in the presence of the recombinant host cell, recombinant bacterium, exosporium fragment, or formulation can exhibit decreased nematode damage, including reduced galling, reduced cysts, and/or reduced nematodes per weight of root, as compared to plants grown in the absence of the enzyme or the microorganism, under the same conditions.

In any of the methods described herein, plants or the locus in which the plant is grown, such as soil, to which the recombinant host cell, recombinant bacterium, exosporium fragment, or formulation has been applied can exhibit reduced nematode eggs and/or reduced nematodes per volume of soil, as compared to plants grown in the absence of the enzyme or the microorganism, under the same conditions.

In one embodiment, the recombinant host cell, recombinant bacterium, exosporium fragment, or formulation of the present invention decreases nematodes and/or nematode damage by at least about 0.5%, or by at least about 1%, or by at least about 2%, or by at least about 3%, or by at least about 5%, or by at least about 6%, or by at least about 7%, or by at least about 8%, or by at least about 9%, or by at least about 10%, or by at least about 11%, or by at least about 12% when compared to plants produced under the same conditions but without treatment.

In any of the methods described herein, plants grown in the presence of the recombinant host cell, recombinant bacterium, exosporium fragment, or formulation can exhibit decreased susceptibility to a pathogen as compared to plants grown in the absence of the enzyme or the microorganism, under the same conditions.

In any of the methods described herein, plants grown in the presence of the recombinant host cell, recombinant bacterium, exosporium fragment, or formulation can exhibit decreased susceptibility to an environmental stress (e.g., drought, flood, heat, freezing, salt, heavy metals, low pH, high pH, or a combination of any thereof) as compared to plants grown in the absence of the enzyme or the microorganism, under the same conditions.

In any of the methods described herein, plants grown in the presence of the recombinant host cell, recombinant bacterium, exosporium fragment, or formulation exhibit can increased root nodulation as compared to plants grown in the absence of the enzyme or the microorganism, under the same conditions.

In any of the methods described herein, plants grown in the presence of the recombinant host cell, recombinant bacterium, exosporium fragment, or formulation can exhibit greater crop yield as compared to plants grown in the absence of the enzyme, or the microorganism, under the same conditions. In one embodiment, the recombinant host cell, recombinant bacterium, exosporium fragment, or formulation of the present invention increases yield or total plant weight by at least about 0.5%, or by at least about 1%, or by at least about 2%, or by at least about 3%, or by at least about 5%, or by at least about 6%, or by at least about 7%, or by at least about 8%, or by at least about 9%, or by at least about 10%, or by at least about 11%, or by at least about 12% when compared to plants produced under the same conditions but without treatment. In another embodiment, the recombinant host cell, recombinant bacterium, exosporium fragment, or formulation of the present invention improves some aspect of plant vigor, such as germination, by at least about 0.5%, or by at least about 1%, or by at least about 2%, or by at least about 3%, or by at least about 5%, or by at least about 6%, or by at least about 7%, or by at least about 8%, or by at least about 9%, or by at least about 10%, or by at least about 11%, or by at least about 12% when compared to plants produced under the same conditions but without treatment.

In any of the methods described herein, plants grown in the presence of the recombinant host cell, recombinant bacterium, exosporium fragment, or formulation can exhibit altered leaf senescence as compared to plants grown in the absence of the enzyme or the microorganism, under the same conditions.

### IX. Carriers

As described above, the formulations described herein comprise an agriculturally acceptable carrier.

The agriculturally acceptable carrier can comprise a dispersant, a surfactant (e.g., a heavy petroleum oil, a heavy petroleum distillate, a polyol fatty acid ester, a polyethoxylated fatty acid ester, an aryl alkyl polyoxyethylene glycol, an alkyl amine acetate, an alkyl aryl sulfonate, a polyhydric alcohol, an alkyl phosphate, or a combination of any thereof), an additive (e.g., an oil, a gum, a resin, a clay, a polyoxyethylene glycol, a terpene, a viscid organic, a fatty acid ester, a sulfated alcohol, an alkyl sulfonate, a petroleum sulfonate, an alcohol sulfate, a sodium alkyl butane diamate, a polyester of sodium thiobutane dioate, a benzene acetonitrile derivative, a proteinaceous material, or a combination of any thereof), water, a thickener (a long chain alkylsulfonate of polyethylene glycol, a polyoxyethylene oleate, or a combination of any thereof), an anti-caking agent (e.g., sodium salt, a calcium carbonate, diatomaceous earth, or a combination of any thereof), a residue breakdown product, a composting formulation, a granular application, diatomaceous earth, an oil, a coloring agent, a stabilizer, a preservative, a polymer, a coating, or a combination of any thereof.

Where the agriculturally acceptable carrier comprises a surfactant, the surfactant can comprise a non-ionic surfactant.

Where the agriculturally acceptable carrier comprises an additive and the additive comprises a proteinaceous material, the proteinaceous material can comprise a milk product, wheat flour, soybean meal, blood, albumin, gelatin, alfalfa meal, yeast extract, or a combination of any thereof.

Where the agriculturally acceptable carrier comprises an anti-caking agent and the anti-caking agent comprises a sodium salt, the sodium salt can comprise a sodium salt of monomethyl naphthalene sulfonate, a sodium salt of dimethyl naphthalene sulfonate, a sodium sulfite, a sodium sulfate, or a combination of any thereof.

The agriculturally acceptable carrier can comprise vermiculite, charcoal, sugar factory carbonation press mud, rice husk, carboxymethyl cellulose, peat, perlite, fine sand, calcium carbonate, flour, alum, a starch, talc, polyvinyl pyrrolidone, or a combination of any thereof.

Any of the formulations described herein can comprise a seed coating formulation (e.g., an aqueous or oil-based solution for application to seeds or a powder or granular formulation for application to seeds), a liquid formulation for application to plants or to a plant growth medium (e.g., a concentrated formulation or a ready-to-use formulation), or a solid formulation for application to plants or to a plant growth medium (e.g., a granular formulation or a powder agent).

The agriculturally acceptable carrier may comprise a formulation ingredient. The formulation ingredient may be a wetting agent, extender, solvent, spontaneity promoter, emulsifier, dispersant, frost protectant, thickener, and/or an adjuvant. In one embodiment, the formulation ingredient is a wetting agent.

Compositions of the present invention may include formulation ingredients added to compositions of the present invention to improve recovery, efficacy, or physical properties and/or to aid in processing, packaging and administration. Such formulation ingredients may be added individually or in combination.

The formulation ingredients may be added to compositions comprising cells, cell-free preparations and/or exosporium fragments to improve efficacy, stability, and physical properties, usability and/or to facilitate processing, packaging and end-use application. Such formulation ingredients may include inerts, stabilization agents, preservatives, nutrients, or physical property modifying agents, which may be added individually or in combination. In some embodiments, the carriers may include liquid materials such as water, oil, and other organic or inorganic solvents and solid materials such as minerals, polymers, or polymer complexes derived biologically or by chemical synthesis. In some embodiments, the formulation ingredient is a binder, adjuvant, or adhesive that facilitates adherence of the composition to a plant part, such as leaves, seeds, or roots. See, for example, Taylor, A. G., et al., “Concepts and Technologies of Selected Seed Treatments,” Annu. Rev. Phytopathol., 28: 321-339 (1990). The stabilization agents may include anti-caking agents, anti-oxidation agents, anti-settling agents, antifoaming agents, desiccants, protectants or preservatives. The nutrients may include carbon, nitrogen, and phosphorus sources such as sugars, polysaccharides, oil, proteins, amino acids, fatty acids and phosphates. The physical property modifiers may include bulking agents, wetting agents, thickeners, pH modifiers, rheology modifiers, dispersants, adjuvants, surfactants, film-formers, hydrotropes, builders, antifreeze agents or colorants. In some embodiments, the composition comprising cells, cell-free preparation and/or exosporium fragments may be used directly with or without water as the diluent without any other formulation preparation. In a particular embodiment, a wetting agent, or a dispersant, is added to a dried concentrate of the whole broth resulting from the fermentation, such as a freeze-dried or spray-dried powder. A wetting agent increases the spreading and penetrating properties, or a dispersant increases the dispersibility and solubility of the active ingredient (once diluted) when it is applied to surfaces. Exemplary wetting agents are known to those of skill in the art and include sulfosuccinates and derivatives, such as MULTIWET™ MO-70R (Croda Inc., Edison, NJ); siloxanes such as BREAK-THRU® (Evonik, Germany); nonionic compounds, such as ATLOX™ 4894 (Croda Inc., Edison, NJ); alkyl polyglucosides, such as TERWET® 3001 (Huntsman International LLC, The Woodlands, Texas); C12-C14 alcohol ethoxylate, such as TERGITOL® 15-S-15 (The Dow Chemical Company, Midland, Michigan); phosphate esters, such as RHODAFAC® BG-510 (Rhodia, Inc.); and alkyl ether carboxylates, such as EMULSOGEN™ LS (Clariant Corporation, North Carolina).

As described above, any of the formulations described herein can comprise an agrochemical.

### X. Plants

In any of the methods described herein relating to plants, the plant can be a dicotyledon, a monocotyledon, a gymnosperm, or an angiosperm.

Likewise, for any of the seeds described herein the seed can be a seed of a dicotyledon, a monocotyledon, a gymnosperm, or an angiosperm.

For example, where the plant is a dicotyledon or the seed is a seed of a dicotyledon, the dicotyledon can be selected from the group consisting of bean, pea, tomato, pepper, squash, alfalfa, almond, aniseseed, apple, apricot, arracha, artichoke, avocado, bambara groundnut, beet, bergamot, black pepper, black wattle, blackberry, blueberry, bitter orange, bok-choi, Brazil nut, breadfruit, broccoli, broad bean, Brussels sprouts, buckwheat, cabbage, camelina, Chinese cabbage, cacao, cantaloupe, caraway seeds, cardoon, carob, carrot, cashew nuts, cassava, castor bean, cauliflower, celeriac, celery, cherry, chestnut, chickpea, chicory, chili pepper, chrysanthemum, cinnamon, citron, citrus, clementine, clove, clover, coffee, cola nut, colza, corn, cotton, cottonseed, cowpea, crambe, cranberry, cress, cucumber, currant, custard apple, drumstick tree, earth pea, eggplant, endive, fennel, fenugreek, fig, filbert, flax, geranium, gooseberry, gourd, grape, grapefruit, guava, hemp, hempseed, henna, hop, horse bean, horseradish, indigo, jasmine, Jerusalem artichoke, jute, kale, kapok, kenaf, kiwi, kohlrabi, kumquat, lavender, lemon, lentil, lespedeza, lettuce, lime, liquorice, litchi, loquat, lupine, macadamia nut, mace, mandarin, mangel, mango, medlar, melon, mint, mulberry, mustard, nectarine, niger seed, nutmeg, okra, olive, opium, orange, papaya, parsnip, pea, peach, peanut, pear, pecan nut, persimmon, pigeon pea, pistachio nut, plantain, plum, pomegranate, pomelo, poppy seed, potato, sweet potato, prune, pumpkin, quebracho, quince, trees of the genus Cinchona, quinoa, radish, ramie, rapeseed, raspberry, rhea, rhubarb, rose, rubber, rutabaga, safflower, sainfoin, salsify, sapodilla, Satsuma, scorzonera, sesame, shea tree, soybean, spinach, squash, strawberry, sugar beet, sugarcane, sunflower, swede, sweet pepper, tangerine, tea, teff, tobacco, tomato, trefoil, tung tree, turnip, urena, vetch, walnut, watermelon, yerba mate, wintercress, shepherd's purse, garden cress, peppercress, watercress, pennycress, star anise, laurel, bay laurel, cassia, jamun, dill, tamarind, peppermint, oregano, rosemary, sage, soursop, pennywort, calophyllum, balsam pear, kukui nut, Tahitian chestnut, basil, huckleberry, hibiscus, passionfruit, star apple, sassafras, cactus, St. John's wort, loosestrife, hawthorn, cilantro, curry plant, kiwi, thyme, zucchini, ulluco, jicama, waterleaf, spiny monkey orange, yellow mombin, starfruit, amaranth, wasabi, Japanese pepper, yellow plum, mashua, Chinese toon, New Zealand spinach, bower spinach, ugu, tansy, chickweed, jocote, Malay apple, paracress, sowthistle, Chinese potato, horse parsley, hedge mustard, campion, agate, cassod tree, thistle, burnet, star gooseberry, saltwort, glasswort, sorrel, silver lace fern, collard greens, primrose, cowslip, purslane, knotgrass, terebinth, tree lettuce, wild betel, West African pepper, yerba santa, tarragon, parsley, chervil, land cress, burnet saxifrage, honeyherb, butterbur, shiso, water pepper, perilla, bitter bean, oca, kampong, Chinese celery, lemon basil, Thai basil, water mimosa, cicely, cabbage-tree, moringa, mauka, ostrich fern, rice paddy herb, yellow sawah lettuce, lovage, pepper grass, maca, bottle gourd, hyacinth bean, water spinach, catscar, fishwort, Okinawan spinach, lotus sweetjuice, gallant soldier, culantro, arugula, cardoon, caigua, mitsuba, chipilin, samphire, mampat, ebolo, ivy gourd, cabbage thistle, sea kale, chaya, huauzontle, Ethiopian mustard, magenta spreen, good king henry, epazole, lamb's quarters, centella plumed cockscomb, caper, rapini, napa cabbage, mizuna, Chinese savoy, kai-lan, mustard greens, Malabar spinach, chard, marshmallow, climbing wattle, China jute, paprika, annatto seed, spearmint, savory, marjoram, cumin, chamomile, lemon balm, allspice, bilberry, cherimoya, cloudberry, damson, pitaya, durian, elderberry, feijoa, jackfruit, jambul, jujube, physalis, purple mangosteen, rambutan, redcurrant, blackcurrant, salal berry, satsuma, ugli fruit, azuki bean, black bean, black-eyed pea, borlotti bean, common bean, green bean, kidney bean, lima bean, mung bean, navy bean, pinto bean, runner bean, mangetout, snap pea, sweet pea, broccoflower, calabrese, nettle, bell pepper, raddichio, daikon, white radish, skirret, tat soi, broccolini, black radish, burdock root, fava bean, broccoli raab, lablab, lupin, sterculia, velvet beans, winged beans, yam beans, mulga, ironweed, umbrella bush, tjuntjula, wakalpulka, witchetty bush, wiry wattle, chia, beech nut, candlenut, colocynth, mamoncillo, Maya nut, mongongo, ogbono nut, paradise nut, and cempedak.

Where the plant is a monocotyledon or the seed is a seed of a monocotyledon, the monocotyledon can be selected from the group consisting of corn, wheat, oat, rice, barley, millet, banana, onion, garlic, asparagus, ryegrass, millet, fonio, raishan, nipa grass, turmeric, saffron, galangal, chive, cardamom, date palm, pineapple, shallot, leck, scallion, water chestnut, ramp, Job's tears, bamboo, ragi, spotless watermeal, arrowleaf elephant ar, Tahitian spinach, abaca, areca, bajra, betel nut, broom millet, broom sorghum, citronella, coconut, cocoyam, maize, dasheen, durra, durum wheat, edo, fique, formio, ginger, orchard grass, esparto grass, Sudan grass, guinea corn, Manila hemp, henequen, hybrid maize, jowar, lemon grass, maguey, bulrush millet, finger millet, foxtail millet, Japanese millet, proso millet, New Zealand flax, oats, oil palm, palm palmyra, sago palm, redtop, sisal, sorghum, spelt wheat, sweet corn, sweet sorghum, sugarcane, taro, teff, timothy grass, triticale, vanilla, wheat, and yam.

Where the plant is a gymnosperm or the seed is a seed of a gymnosperm, the gymnosperm can be from a family selected from the group consisting of Araucariaccac, Boweniaceae, Brassicaceac, Cephalotaxaceac, Cupressaceae, Cycadaceac, Ephedraceac, Ginkgoaccac, Gnetaccac, Pinaceae, Podocarpaceae, Taxaccac, Taxodiaceae, Welwitschiaccac, and Zamiaccac.

The plants and plant seeds described herein may include transgenic plants or plant seeds, such as transgenic cereals (wheat, rice), maize, soybean, potato, cotton, tobacco, oilseed rape and fruit plants (fruit of apples, pears, citrus fruits and grapes, including wine grapes). Preferred transgenic plants include corn, soybeans, potatoes, cotton, tobacco, sugar beet, sugarcane, and oilseed rape.

Plant seeds as described herein can be genetically modified (e.g., any seed that results in a genetically modified plant or plant part that expresses herbicide tolerance, tolerance to environmental factors such as water stress, drought, viruses, and nitrogen production, or resistance to bacterial, fungi or insect toxins). Suitable genetically modified seeds include those of cole crops, vegetables, fruits, trees, fiber crops, oil crops, tuber crops, coffee, flowers, legume, cereals, as well as other plants of the monocotyledonous and dicotyledonous species. Preferably, the genetically modified seeds include peanut, tobacco, grasses, wheat, barley, rye, sorghum, rice, rapeseed, sugarbeet, sunflower, tomato, pepper, bean, lettuce, potato, and carrot. Most preferably, the genetically modified seeds include cotton, soybean, and corn (sweet, field, seed, or popcorn).

Particularly useful transgenic plants which may be treated according to the invention are plants containing transformation events, or a combination of transformation events, that are listed for example in the databases from various national or regional regulatory agencies (see for example www.gmoinfo.jrc.it/gmp_browse.aspx and www.agbios.com/dbase.php).

Having described the invention in detail, it will be apparent that modifications and variations are possible without departing from the scope of the invention defined in the appended claims.

### EXAMPLES

### Example 1

### Assays for Serine Protease Activity of Sep1 or Sep1 Variants in Bacillus cereus

1. Construction of a Bacillus cereus Family Member Displaying a Serine Protease or Serine Protease Variant

To evaluate the serine protease activity of wild-type and variant serine proteases, Bacillus cereus family members displaying the serine protease of SEQ ID NO: 1 or the serine protease variant of SEQ ID NO: 2 were constructed. The pSUPER plasmid was generated through fusion of the pUC57 plasmid (containing an ampicillin resistance cassette and a ColE1 origin of replication) with the pBC16-1 plasmid from Bacillus cereus (containing a tetracycline resistance gene, repU replication gene and oriU origin of replication). This 5.8 kb plasmid can replicate in both E. coli and Bacillus spp. and can be selected by conferring resistance to β-lactam antibiotics in E. coli and resistance to tetracycline in Bacillus spp. The basal pSUPER plasmid was modified by insertion of a PCR-generated fragment that fused a promoter, a start codon, a targeting sequence, and an alanine linker sequence in frame with SEQ ID NO: 1 or SEQ ID NO: 2, resulting in pSUPER plasmids. These constructs were transformed into E. coli and plated on Lysogeny broth plates plus ampicillin (100 μg/mL) to obtain single colonies. Individual colonies were used to inoculate Lysogeny broth plus ampicillin and incubated overnight at 37° C. 300 rpm. Plasmids from resulting cultures were extracted using a commercial plasmid purification kit. DNA concentrations of these plasmid extracts were determined via spectrophotometry, and obtained plasmids subjected to analytical digests with appropriate combinations of restriction enzymes. The resulting digestion patterns were visualized by agarose gel electrophoresis to investigate plasmid size and presence of distinct plasmid features. Relevant sections, such as the SEQ ID NO: 1 or SEQ ID NO: 2 expression cassette, of the purified pSUPER derivatives were further investigated by Sanger sequencing.

Additionally, a derivative plasmid of the pSUPER plasmids described above was created as follows. The pBC fragment (pBC16-1-derived section of pSUPER including BclA/serine protease variant expression cassette and tetracycline resistance) of the pSUPER plasmids described above was amplified by PCR and subsequently circularized by blunt-end ligation.

pSUPER, verified as described above, and pBC plasmid ligations were introduced by electroporation into Bacillus thuringiensis BT013A (Accession No. NRRL B-50924). Single transformed colonies were isolated by plating on nutrient broth plates containing tetracycline (10 μg/mL). Individual positive colonies were used to inoculate brain heart infusion broth containing tetracycline (10 μg/mL) and incubated overnight at 30° ° C., 300 rpm. Genomic DNA of resulting cultures was purified and relevant sections of the pSUPER plasmid or the pBC plasmid were re-sequenced to confirm genetic purity of the cloned sequences and, for pBC, the correct ligation site. Verified colonies were grown overnight in brain heart infusion broth with 10 μg/mL tetracycline and induced to sporulate through incubation in a yeast extract-based media at 30° ° C. for 48 hours.

2. Construction and Purification of a Knockout Mutant Strain of Bacillus thuringiensis Expressing Serine Protease Variant

To make exsY knockout (KO) mutant strains of Bacillus thuringiensis BT013A, the plasmid pKOKI shuttle and integration vector was constructed that contained the pUC57 backbone, which is able to replicate in E. coli, as well as the origin of replication and the erythromycin resistance cassette from pE194. This construct is able to replicate in both E. coli and Bacillus spp. A construct was made that contained the 1 kb DNA region that corresponded to the upstream region of the exsY gene and a 1 kb region that corresponded to the downstream region of the gene exsY, both of which were PCR amplified from Bacillus thuringiensis BT013A. For each construct, the two 1 kb regions were then spliced together using homologous recombination with overlapping regions to each other and with the pKOKI plasmid, respectively. This plasmid construct was verified by digestion and DNA sequencing. Clones were screened for erythromycin resistance.

Clones were passaged under high temperature (40° C.) in brain heart infusion broth. Individual colonies were toothpicked onto LB agar plates containing erythromycin 5 μg/mL, grown at 30° ° C., and screened for the presence of the pKOKI plasmid integrated into the chromosome by colony PCR. Colonies that had an integration event were continued through passaging to screen for single colonies that lost erythromycin resistance (signifying loss of the plasmid by recombination and removal of the exsY gene). Verified deletions were confirmed by PCR amplification and sequencing of the target region of the chromosome. Finally, the PCR-amplified, circularized pBC section of the pSUPER plasmids (described above) was transformed into this exsY mutant strain of BT013A.

For each esxYKO mutant expressing the serine protease of SEQ ID NO: 1 or the serine protease variant of SEQ ID NO: 2, an overnight culture was grown in BHI media at 30° C. 300 rpm, in baffled flasks with antibiotic selection. One milliliter of this overnight culture was inoculated into a yeast extract-based media (50 mL) in a baffled flask and grown at 30° C. for 2 days. An aliquot of spores was removed and the spores were agitated by vortexing. The spores were collected via centrifugation at 8,000×g for 10 minutes, and supernatant containing the exosporium fragments was filtered through a 0.22 μm filter to remove any residual spores. No spores were found in the filtrate.

### 3. Activity of Serine Protease Variants

Whole broth cultures of esxYKO mutants without a recombinant plasmid (9.79×10⁷ CFU/mL), or expressing the serine protease of SEQ ID NO: 1 (7.06×10⁷ CFU/mL) or SEQ ID NO: 2 (7.06×10⁷ CFU/mL) were grown to the CFU concentrations designated after each strain. Exosporium fragment filtrates were generated, and equal volumes of each were tested as follows. Enzyme activity was determine using synthetic peptide substrate (Ala-Ala-Pro-Phe). The peptide substrate is fused with nitro phenyl at the C-terminus and succinyl group at the N-terminus. The peptide shows absorbance maxima at 320 nm before protease cleavage and shifts to 390 nm following the cleavage. The assay mixture consisted of 10 μL of 2.5 mg/mL peptide substrate in 240 μL of 50 mM Hepes buffer pH 7.5, containing 5 mM CaCl₂). The substrate and the buffer were pre-incubated at room temperature, followed by the addition of 25 μL of the enzyme solution. Results are shown in FIG. 1. This data shows that both whole broth cultures have enzymatic activity and that the strain comprising serine protease variant SEQ ID NO: 2 has slightly higher activity than does the strain comprising SEQ ID NO: 1. Without wishing to be bound by any theory, Applicant hypothesizes that the recombinant Bacillus cereus family member with the variant serine protease (SEQ ID NO: 2) has greater biological activity than the recombinant Bacillus cereus family member with the full-length serine protease (SEQ ID NO: 1). Also without wishing to be bound by theory, it is hypothesized that the deletion within the serine protease variant of SEQ ID NO: 2 may alter or improve serine protease activity by making the binding groove corresponding to residues 122-126 and 146-151 of SEQ ID NO: 1 more accessible to the substrate.

### Example 2

### Computer Modeling of Serine Protease Variants

In order to further investigate the effect of sequence modification on serine protease activity, additional serine protease variants were developed and tested. Wild-type and variant sequences used in this study to investigate the effect of structural feature removal or deliberate perturbations to the catalytic triad are shown in Table 1.

TABLE 1
Variants of the Sep1 serine protease of Bacillus firmus used in homology modeling.

| SEQ ID | | |
|---|---|---|
| NO. | Sequence Name | Description |
| 1 | W7KRH1_BACFI | Wild-Type Bacillus firmus DS-1 Intracellular Serine |
| | | Protease (UniProt Accession No. W7KRH1_BACFI) |
| 2 | SEP1_trunc | Variant of serine protease from Bacillus firmus strain DS- |
| | | 1 (Sep1 truncation). Deletion of residues 181-240 of SEQ |
| | | ID NO: 1. |
| 3 | IMEE | Homologous template from Bacillus pumilus (UniProt |
| | | Accession No. P07518 (SUBT_BACPU)) |
| 4 | Intermediate_deletion_1 | Deletion of residues 226-242 of SEQ ID NO: 1 |
| 5 | Intermediate_deletion_2 | Deletion of residues 212-241 of SEQ ID NO: 1. |
| 6 | Intermediate_deletion_3 | Deletion of residues 182-211 of SEQ ID NO: 1. |
| 7 | Extended_deletion_1 | Deletion of residues 181-243 of SEQ ID NO: 1 |
| 8 | Extended_deletion_2 | Deletion of residues 178-240 of SEQ ID NO: 1. |
| 9 | Extended_deletion_3 | Deletion of residues 178-243 of SEQ ID NO: 1 |
| 10 | Extended_deletion_4 | Deletion of residues 177-243 of SEQ ID NO: 1 with loss |
| | | of N at residue 177 of SEQ ID NO: 1. |
| 11 | Extended_deletion_5 | Deletion of residues 176-243 of SEQ ID NO: 1 with loss |
| | | of N at residue 177 of SEQ ID NO: 1. |
| 12 | Extended_deletion_6 | Deletion of residues 175-243 of SEQ ID NO: 1 with loss |
| | | of N at residue 177 of SEQ ID NO: 1. |
| 13 | Extended_deletion_7 | Deletion of residues 174-243 of SEQ ID NO: 1 with loss |
| | | of N at residue 177 of SEQ ID NO: 1. |

SEQ ID NO: 1 is the wild-type Sep1 serine protease from Bacillus firmus strain DS-1. SEQ ID NO: 2 is a variant of SEQ ID NO: 1 constructed by deleting amino acids 181-240 of SEQ ID NO: 1. As shown in Example 1 above. SEQ ID NO: 2 exhibits serine protease activity. SEQ ID NOs: 4-6 represent intermediate deletions of SEQ ID NO: 1. SEQ ID NOs: 7-13 represent extended deletions of SEQ ID NO: 1, in which additional residues were deleted compared with SEQ ID NO: 2.

For modeling studies, a homologous serine protease from Bacillus pumilus (UnitProt Accession No. P07518 (SUBT_BACPU); SEQ ID NO: 3) was selected as a homology template. The template was chosen based on sequence similarity and the high degree of structural feature conservation among serine proteases generally. As shown in FIG. 2, the template sequence of SEQ ID NO: 3 shows a significant degree of amino acid residue type similarity between the aligned sequences. For example, polar residues are aligned with different polar residues.

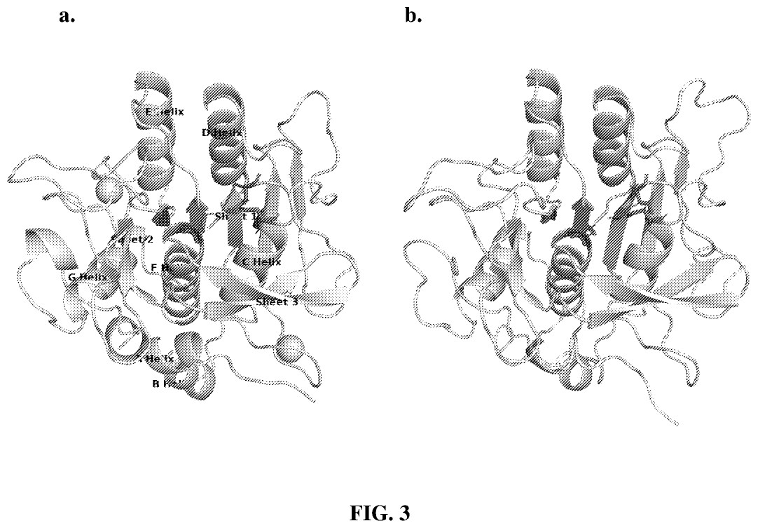

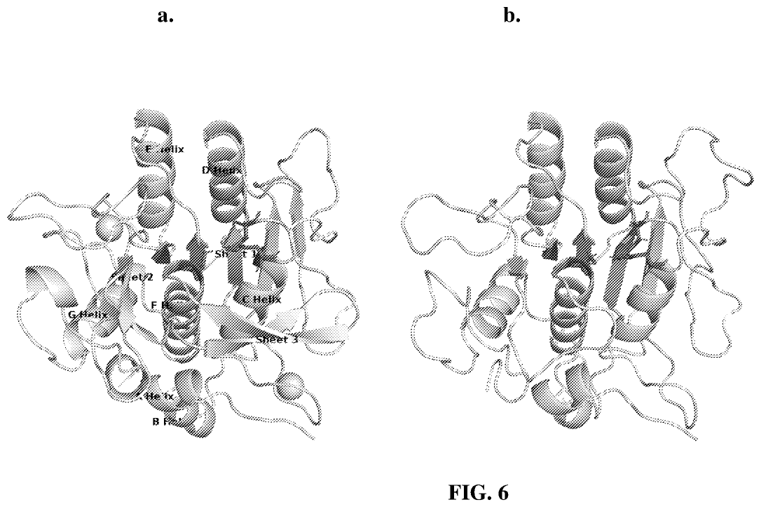

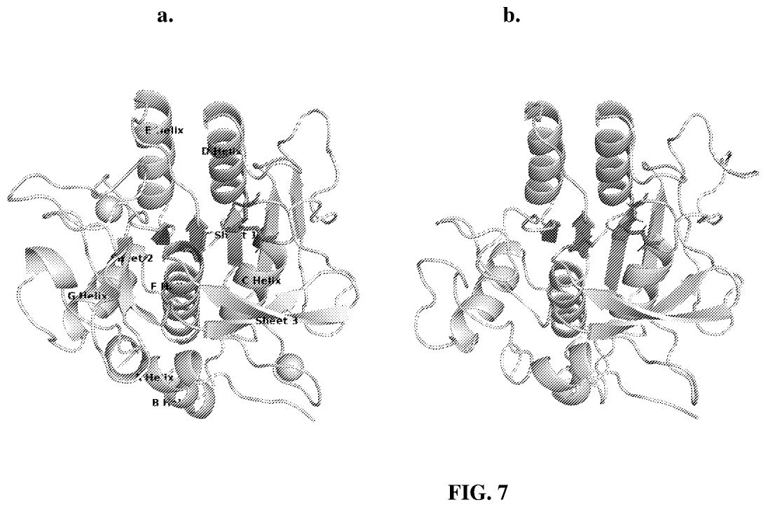

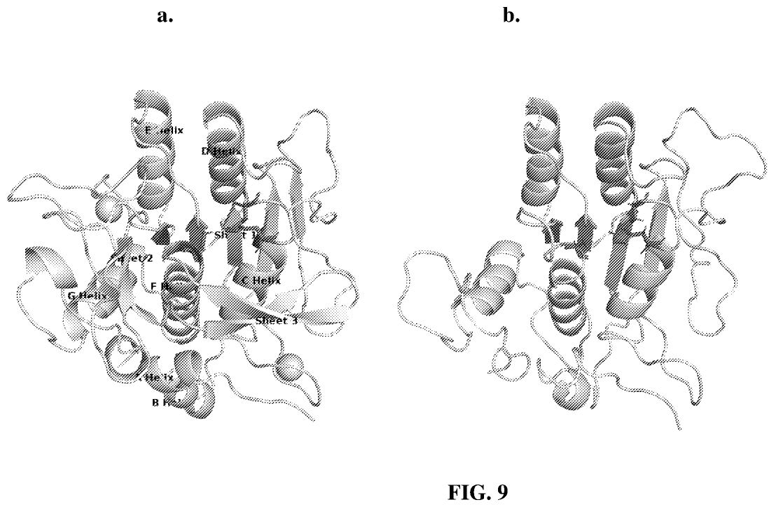

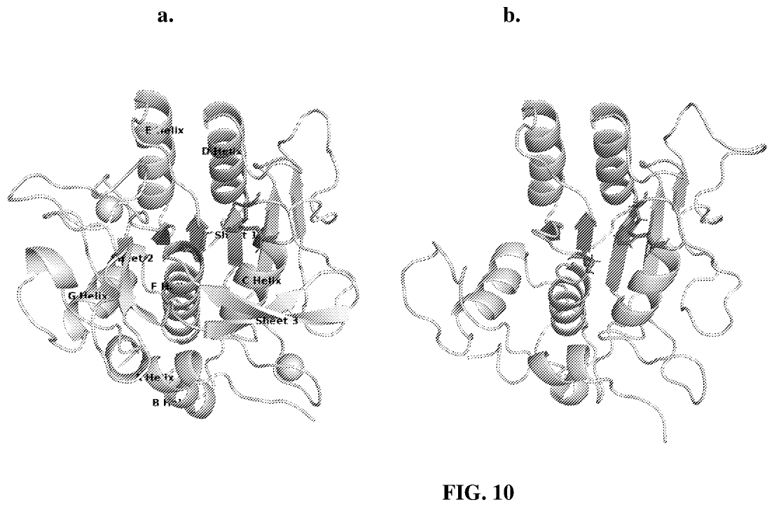

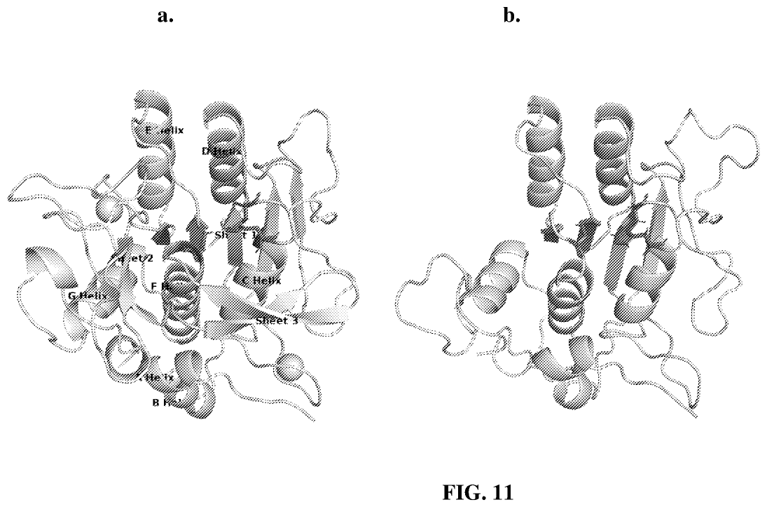

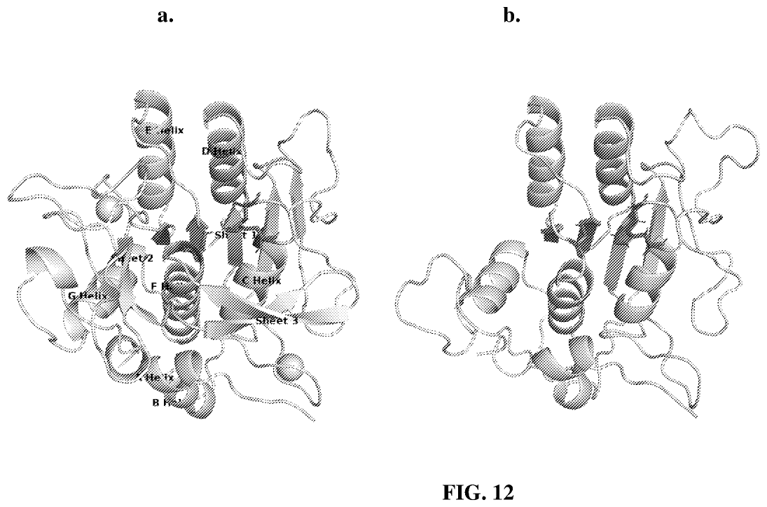

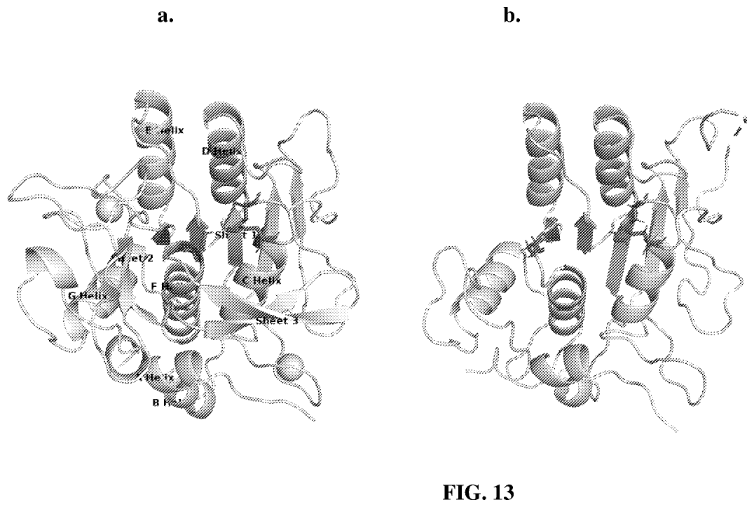

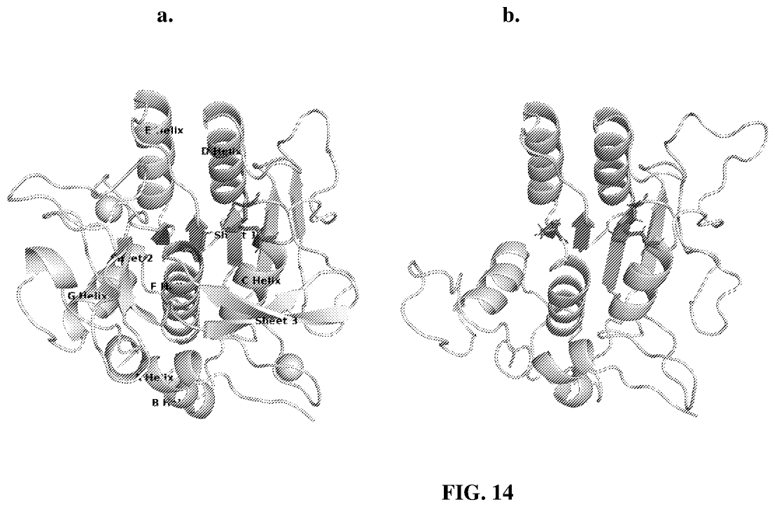

In addition, as shown in the multiple sequence alignment in FIG. 2, key features of wild-type serine proteases are conserved in the catalytic triad and binding regions. For example, residues corresponding to Asp49, His86, and Ser244 of SEQ ID NO: 1 forming the catalytic triad in wild-type sequences (identified with rectangles in FIGS. 2a, 2b, and 2c) are conserved in each of the modeled variants. Additionally, the substrate binding region corresponding to Gly122, Ser123, Gly124, Gln125, and Tyr126 and Met146, Ser147, Leu148, Gly149, Gly150, Pro151 of SEQ ID NO: 1 (identified with rectangles in FIG. 2c) is completely conserved between modeled variants and SEQ ID NO: 3 on the basis of the multiple sequence alignment. Furthermore, conserved residues within the catalytic triad and binding groove retain their general geometric positions as shown in the models in FIG. 3-14.

### Example 3

### Conservation of Secondary Structure Between Serine Protease Variants

Sequences were modeled using the Triad modeling suite (Protabit, LLC) with the 1MEE PDB entry for Bacillus pumilus (UnitProt Accession No. P07518 (SUBT_BACPU); SEQ ID NO: 3) as the modeling template. Template-based homology modeling with standard refinement settings was performed. Specifically, the software was allowed to adjust the random atomic perturbation radius in each modeling cycle. Additionally, six weighted restraint cycles to conserve template features were performed with weights of [1.0, 0.001, 0.75, 0.001, 0.25, 0.0], thus “shaking” the structure toward the template and then allowing it to relax away from the template. A trim error threshold of [1.0] was used given a template was provided for modeling that is reasonable in its sequence similarity and as it derives from phylogenetically related species. Neither reference alignment, symmetry restraints, nor unaligned sequence trimming was performed. The best scoring model was selected for output. No further analysis in Triad was performed. The resultant structures were visualized, aligned, and analyzed using PyMol (Schroedinger).

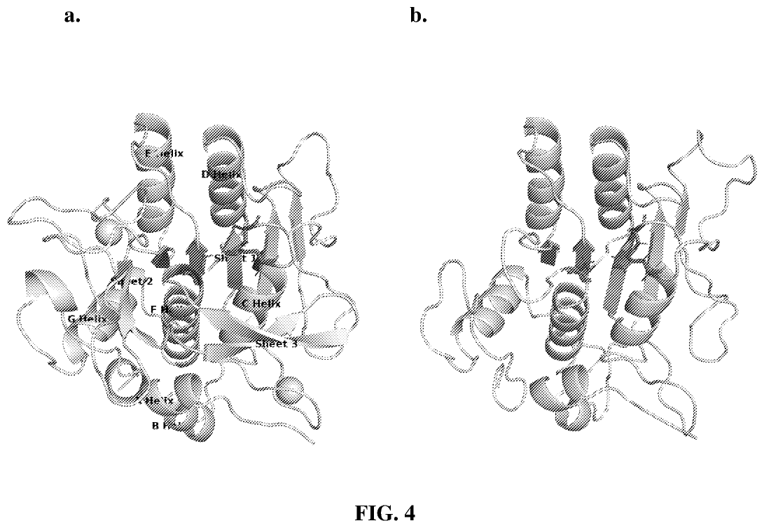

As shown in FIG. 3, homology modeling indicates strong structural conservation between the template of SEQ ID NO: 3 and the wild-type (SEQ ID NO: 1). The structure is also conserved between template SEQ ID NO: 3 and variant serine protease SEQ ID NO: 2, as shown in FIG. 4.

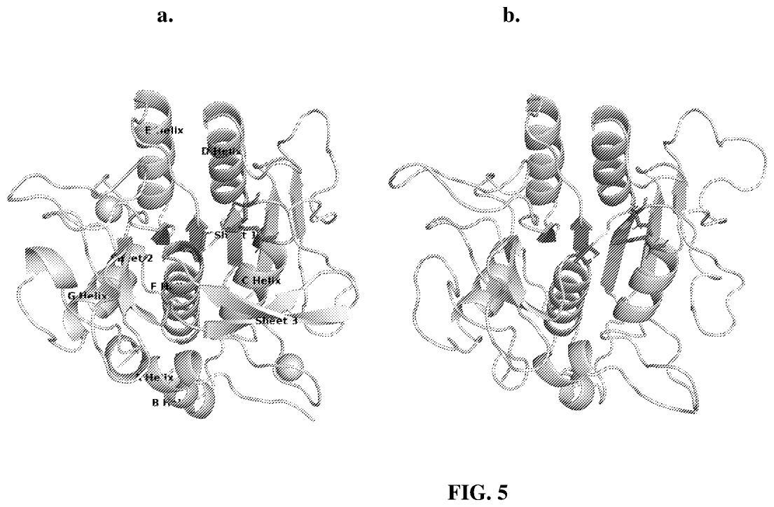

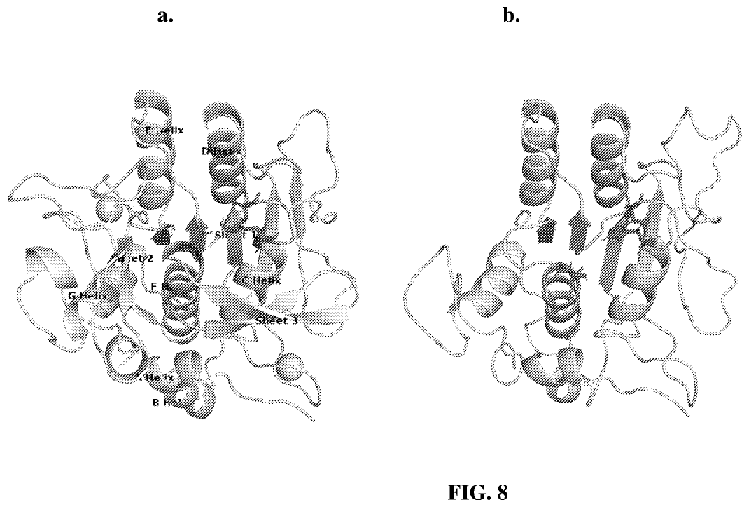

FIG. 5-7 show structural conservation between all intermediate deletion variants tested (SEQ ID NO: 4-6) and the template sequence (SEQ ID NO: 3). As shown in FIG. 8-13, all extended deletion variants are also predicted to maintain similar structure, particularly within the conserved catalytic triad and the binding region.

### Example 4

### Analysis of Catalytic Triad Geometry in Sep1 Variants

Charge relay is required for serine protease enzymatic activity, and significant distortion of the catalytic triad will disrupt charge relay and catalytic competence. Following homology modeling and in order to investigate the geometry of the catalytic triad in the serine protease variants, the alpha carbon distance versus the 1MEE homology template (SEQ ID NO: 3) was calculated for each residue of the catalytic triad. Root mean square deviations (RMSD) for the triad compared with 1MEE were also calculated. Variants having a triad RMSD of approximately 1 Å exhibit substantial structural similarity and are likely to retain charge relay function and enzymatic activity in light of sequence alignment and general conservation of secondary structures in the generated model compared to the known active template.

TABLE 2
Analysis of catalytic triad geometry of serine protease variants.

| | SEQ | Triad Cα Distance or RMSD vs 1MEE (Å) | | | |
|---|---|---|---|---|---|
| Entry | ID NO. | Serine | Aspartate | Histidine | Triad RMSD |
| 1MEE | 3 | 0 | 0 | 0 | 0 |
| W7KRH1_BACFI | 1 | 0.47 | 0.2 | 0.58 | 0.45 |
| Intermediate_deletion_3 | 6 | 0.57 | 0.21 | 0.88 | 0.62 |
| Extended_deletion_2 | 8 | 0.9 | 0.19 | 0.78 | 0.70 |
| Extended_deletion_4 | 10 | 0.91 | 0.4 | 0.82 | 0.74 |
| Intermediate_deletion_2 | 5 | 0.64 | 0.6 | 0.98 | 0.76 |
| SEP1_trunc | 2 | 0.39 | 0.47 | 1.19 | 0.77 |
| Intermediate_deletion_1 | 4 | 0.82 | 1.04 | 1.14 | 1.01 |
| Extended_deletion_3 | 9 | 2.12 | 0.4 | 0.92 | 1.35 |
| Extended_deletion_1 | 7 | 3.01 | 0.44 | 0.65 | 1.80 |
| Extended_deletion_5 | 11 | 3.49 | 0.37 | 0.72 | 2.07 |
| Extended_deletion_7 | 13 | 5 | 0.19 | 0.69 | 2.92 |
| Extended_deletion_6 | 12 | 5.93 | 0.21 | 0.57 | 3.44 |

As shown in Table 2, wild-type serine protease (W7KRH1_BACFI; SEQ ID NO: 1) showed minimal perturbation of the residues making up the catalytic triad. Similarly, each of the intermediate deletion variants (SEQ ID NOs: 4-6) maintained structural similarity to the homology template. The truncation variant (SEQ ID NO: 2), as well as Extended_deletion_2 (SEQ ID NO: 8) and Extended_deletion_4 (SEQ ID NO: 10) also exhibited minimal perturbation to the catalytic triad. Extended_deletion_3 (SEQ ID NO: 9) exhibited moderate perturbation to the catalytic triad, while Extended_deletion_1 (SEQ ID NO: 7), Extended_deletion_5 (SEQ ID NO: 11), Extended_deletion_7 (SEQ ID NO: 13), and Extended_deletion_6 (SEQ ID NO: 12) exhibited RMSD of 1.80 Å or greater.

These data demonstrate that SEQ ID NO: 2, shown to have serine protease activity in Example 1, maintains structural similarity in terms of gross secondary features, in particular within the binding groove of the protein and with respect to positioning of the catalytic triad. Similarly, minimal perturbation of the structure is observed with respect to the relative geometry of catalytic triad alpha carbons for shorter and intermediate deletion variants (SEQ ID NOs: 4-6 and 8-10).

Deletions of more than 7 additional residues compared with SEQ ID NO: 2 in the direction of the N-terminus appear to experience more significant geometric perturbation to the catalytic triad, specifically at the serine residue. In addition, deletion of 7 additional residues compared with SEQ ID NO: 2 toward the N-terminus from the catalytic serine deletes the asparagine at position 177 of SEQ ID NO: 2. Asn177 is positioned to participate in the oxyanionic hole in the active site during catalysis, and disruption of this residue may decrease catalytic competency.

1. A recombinant DNA molecule comprising a nucleic acid sequence encoding a polypeptide with serine protease activity, wherein said polypeptide comprises an aspartic acid at a residue corresponding to residue 49 of SEQ ID NO: 1, a histidine at a residue corresponding to residue 86 of SEQ ID NO: 1, and a serine at a residue corresponding to residue 244 of SEQ ID NO: 1, or a conservative substitution of any of these three residues, and wherein the polypeptide comprises at least a first amino acid residue deletion relative to SEQ ID NO: 1.

2. The recombinant DNA molecule of claim 1, wherein the encoded polypeptide comprises an amino acid deletion of at least one residue corresponding to any of residues 177-243 of SEQ ID NO: 1.

3. The recombinant DNA molecule of claim 2, wherein the encoded polypeptide comprises an amino acid deletion of at least one residue corresponding to any of residues 181-240 of SEQ ID NO: 1.

4. The recombinant DNA molecule of claim 1, wherein the encoded polypeptide comprises an amino acid deletion of at least the residues corresponding to residues 226-241 of SEQ ID NO: 1.

5. The recombinant DNA molecule of claim 1, wherein the encoded polypeptide comprises an amino acid deletion of at least the residues corresponding to residues 182-211 of SEQ ID NO: 1.

6. The recombinant DNA molecule of claim 1, wherein the encoded polypeptide comprises an amino acid deletion of at least the residues corresponding to residues 178-243 of SEQ ID NO: 1.

7-8. (canceled)

9. The recombinant DNA molecule of claim 1, wherein the encoded polypeptide exhibits increased nematicidal activity or increased antifungal activity compared to the polypeptide of SEQ ID NO: 1.

10. (canceled)

11. The recombinant DNA molecule of claim 1, wherein the encoded polypeptide comprises a glycine at a residue corresponding to residue 122 of SEQ ID NO: 1, a serine at a residue corresponding to residue 123 of SEQ ID NO: 1, a glycine at a residue corresponding to residue 124 of SEQ ID NO: 1, a glutamine at a residue corresponding to residue 125 of SEQ ID NO: 1, a tyrosine at a residue corresponding to residue 126 of SEQ ID NO: 1, a methionine at a residue corresponding to residue 146 of SEQ ID NO: 1, a serine at a residue corresponding to residue 147 of SEQ ID NO: 1, a leucine at a position corresponding to residue 148 of SEQ ID NO: 1, a glycine at a residue corresponding to residue 149 of SEQ ID NO: 1, a glycine at a residue corresponding to residue 150 of SEQ ID NO: 1, and a proline at a residue corresponding to residue 151 of SEQ ID NO: 1, or a conservative substitution of any of these residues.

12. A polypeptide encoded by the recombinant DNA molecule of any one of claims 1-11.

13. (canceled)

14. A DNA construct comprising the recombinant DNA molecule of claim 1 operably linked to a promoter.

15. A host cell comprising the recombinant DNA molecule of claim 1.

16. The host cell of claim 15, wherein the host cell is a bacterial host cell.

17-20. (canceled)

21. A method for stimulating plant growth and/or promoting plant health and/or controlling nematodes, comprising applying the recombinant host cell of claim 15 to a plant growth medium, a plant, a plant seed, or an area surrounding a plant or a plant seed.

22. A fusion protein comprising:
a) a targeting sequence, exosporium protein, or exosporium protein fragment that targets the fusion protein to the exosporium of a recombinant Bacillus host cell; and
b) the polypeptide of claim 12.

23. A recombinant host cell that expresses the fusion protein of claim 22.

24. The host cell of claim 23, wherein the host cell is a bacterial host cell.

25. The host cell of claim 24, wherein the bacterial host cell is from a Bacillus cereus family member.

26. A fermentation product of the bacterial host cell of claim 23.

27. A formulation comprising the fermentation product of claim 26 and an agriculturally acceptable carrier.

28. A method for stimulating plant growth and/or promoting plant health and/or controlling nematodes, comprising applying the formulation of claim 27 to a plant growth medium, a plant, a plant seed, or an area surrounding a plant or a plant seed.


SEQUENCE LISTING (14 sequences)

SEQ ID NO: 1 - protein, 321 aa, Bacillus firmus
MEQYVRVIPYKVIQQEENVKEVPKGVELIQAPKVWSETKGKGIKIAVLDTGCDISHPDLKDRVTGGRNFTDDDNSDPNSFKDYNGHGTHVAGTIAAYENNAGVIGVAPEAELLIVKVLNKDGSGQYEWIIKGIHYAIEQNADIISMSLGGPADVPELHDAIKAAVNKNILVVCAAGNEGDGDDSTDEFAYPGCYNEVISVGAINLERDSSDFTNSHNEIDLVAPGEGILSTFLNGKYATLSGTSMAAPHVSGALALIKDFANRQFERKLSEPELYAQLIRRTVPLGNSPKLEGNGLVYLTVPDHLAGIFDQELKSTVLNAI

SEQ ID NO: 2 - protein, 261 aa, Artificial Sequence
  Recombinant polypeptide
MEQYVRVIPYKVIQQEENVKEVPKGVELIQAPKVWSETKGKGIKIAVLDTGCDISHPDLKDRVTGGRNFTDDDNSDPNSFKDYNGHGTHVAGTIAAYENNAGVIGVAPEAELLIVKVLNKDGSGQYEWIIKGIHYAIEQNADIISMSLGGPADVPELHDAIKAAVNKNILVVCAAGNEGDSGTSMAAPHVSGALALIKDFANRQFERKLSEPELYAQLIRRTVPLGNSPKLEGNGLVYLTVPDHLAGIFDQELKSTVLNAI

SEQ ID NO: 3 - protein, 275 aa, Bacillus pumilus
AQSVPYGISQIKAPALHSQGYTGSNVKVAVIDSGIDSSHPDLNVRGGASFVPSETNPYQDGSSHGTHVAGTIAALNNSIGVLGVAPSSALYAVKVLDSTGSGQYSWIINGIEWAISNNMDVINMSLGGPTGSTALKTVVDKAVSSGIVVAAAAGNEGSSGSTSTVGYPAKYPSTIAVGAVNSANQRASFSSAGSELDVMAPGVSIQSTLPGGTYGAYNGTSMATPHVAGAAALILSKHPTWTNAQVRDRLESTATYLGSSFYYGKGLINVQAAAQ

SEQ ID NO: 4 - protein, 304 aa, Artificial Sequence
  Recombinant polypeptide
MEQYVRVIPYKVIQQEENVKEVPKGVELIQAPKVWSETKGKGIKIAVLDTGCDISHPDLKDRVTGGRNFTDDDNSDPNSFKDYNGHGTHVAGTIAAYENNAGVIGVAPEAELLIVKVLNKDGSGQYEWIIKGIHYAIEQNADIISMSLGGPADVPELHDAIKAAVNKNILVVCAAGNEGDGDDSTDEFAYPGCYNEVISVGAINLERDSSDFTNSHNEIDLVAPGTSMAAPHVSGALALIKDFANRQFERKLSEPELYAQLIRRTVPLGNSPKLEGNGLVYLTVPDHLAGIFDQELKSTVLNAI

SEQ ID NO: 5 - protein, 291 aa, Artificial Sequence
  Recombinant polypeptide
MEQYVRVIPYKVIQQEENVKEVPKGVELIQAPKVWSETKGKGIKIAVLDTGCDISHPDLKDRVTGGRNFTDDDNSDPNSFKDYNGHGTHVAGTIAAYENNAGVIGVAPEAELLIVKVLNKDGSGQYEWIIKGIHYAIEQNADIISMSLGGPADVPELHDAIKAAVNKNILVVCAAGNEGDGDDSTDEFAYPGCYNEVISVGAINLERDSSDGTSMAAPHVSGALALIKDFANRQFERKLSEPELYAQLIRRTVPLGNSPKLEGNGLVYLTVPDHLAGIFDQELKSTVLNAI

SEQ ID NO: 6 - protein, 291 aa, Artificial Sequence
  Recombinant polypeptide
MEQYVRVIPYKVIQQEENVKEVPKGVELIQAPKVWSETKGKGIKIAVLDTGCDISHPDLKDRVTGGRNFTDDDNSDPNSFKDYNGHGTHVAGTIAAYENNAGVIGVAPEAELLIVKVLNKDGSGQYEWIIKGIHYAIEQNADIISMSLGGPADVPELHDAIKAAVNKNILVVCAAGNEGDGFTNSHNEIDLVAPGEGILSTFLNGKYATLSGTSMAAPHVSGALALIKDFANRQFERKLSEPELYAQLIRRTVPLGNSPKLEGNGLVYLTVPDHLAGIFDQELKSTVLNAI

SEQ ID NO: 7 - protein, 258 aa, Artificial Sequence
  Recombinant polypeptide
MEQYVRVIPYKVIQQEENVKEVPKGVELIQAPKVWSETKGKGIKIAVLDTGCDISHPDLKDRVTGGRNFTDDDNSDPNSFKDYNGHGTHVAGTIAAYENNAGVIGVAPEAELLIVKVLNKDGSGQYEWIIKGIHYAIEQNADIISMSLGGPADVPELHDAIKAAVNKNILVVCAAGNEGDSMAAPHVSGALALIKDFANRQFERKLSEPELYAQLIRRTVPLGNSPKLEGNGLVYLTVPDHLAGIFDQELKSTVLNAI

SEQ ID NO: 8 - protein, 258 aa, Artificial Sequence
  Recombinant polypeptide
MEQYVRVIPYKVIQQEENVKEVPKGVELIQAPKVWSETKGKGIKIAVLDTGCDISHPDLKDRVTGGRNFTDDDNSDPNSFKDYNGHGTHVAGTIAAYENNAGVIGVAPEAELLIVKVLNKDGSGQYEWIIKGIHYAIEQNADIISMSLGGPADVPELHDAIKAAVNKNILVVCAAGNSGTSMAAPHVSGALALIKDFANRQFERKLSEPELYAQLIRRTVPLGNSPKLEGNGLVYLTVPDHLAGIFDQELKSTVLNAI

SEQ ID NO: 9 - protein, 255 aa, Artificial Sequence
  Recombinant polypeptide
MEQYVRVIPYKVIQQEENVKEVPKGVELIQAPKVWSETKGKGIKIAVLDTGCDISHPDLKDRVTGGRNFTDDDNSDPNSFKDYNGHGTHVAGTIAAYENNAGVIGVAPEAELLIVKVLNKDGSGQYEWIIKGIHYAIEQNADIISMSLGGPADVPELHDAIKAAVNKNILVVCAAGNSMAAPHVSGALALIKDFANRQFERKLSEPELYAQLIRRTVPLGNSPKLEGNGLVYLTVPDHLAGIFDQELKSTVLNAI

SEQ ID NO: 10 - protein, 254 aa, Artificial Sequence
  Recombinant polypeptide
MEQYVRVIPYKVIQQEENVKEVPKGVELIQAPKVWSETKGKGIKIAVLDTGCDISHPDLKDRVTGGRNFTDDDNSDPNSFKDYNGHGTHVAGTIAAYENNAGVIGVAPEAELLIVKVLNKDGSGQYEWIIKGIHYAIEQNADIISMSLGGPADVPELHDAIKAAVNKNILVVCAAGSMAAPHVSGALALIKDFANRQFERKLSEPELYAQLIRRTVPLGNSPKLEGNGLVYLTVPDHLAGIFDQELKSTVLNAI

SEQ ID NO: 11 - protein, 253 aa, Artificial Sequence
  Recombinant polypeptide
MEQYVRVIPYKVIQQEENVKEVPKGVELIQAPKVWSETKGKGIKIAVLDTGCDISHPDLKDRVTGGRNFTDDDNSDPNSFKDYNGHGTHVAGTIAAYENNAGVIGVAPEAELLIVKVLNKDGSGQYEWIIKGIHYAIEQNADIISMSLGGPADVPELHDAIKAAVNKNILVVCAASMAAPHVSGALALIKDFANRQFERKLSEPELYAQLIRRTVPLGNSPKLEGNGLVYLTVPDHLAGIFDQELKSTVLNAI

SEQ ID NO: 12 - protein, 252 aa, Artificial Sequence
  Recombinant polypeptide
MEQYVRVIPYKVIQQEENVKEVPKGVELIQAPKVWSETKGKGIKIAVLDTGCDISHPDLKDRVTGGRNFTDDDNSDPNSFKDYNGHGTHVAGTIAAYENNAGVIGVAPEAELLIVKVLNKDGSGQYEWIIKGIHYAIEQNADIISMSLGGPADVPELHDAIKAAVNKNILVVCASMAAPHVSGALALIKDFANRQFERKLSEPELYAQLIRRTVPLGNSPKLEGNGLVYLTVPDHLAGIFDQELKSTVLNAI

SEQ ID NO: 13 - protein, 251 aa, Artificial Sequence
  Recombinant polypeptide
MEQYVRVIPYKVIQQEENVKEVPKGVELIQAPKVWSETKGKGIKIAVLDTGCDISHPDLKDRVTGGRNFTDDDNSDPNSFKDYNGHGTHVAGTIAAYENNAGVIGVAPEAELLIVKVLNKDGSGQYEWIIKGIHYAIEQNADIISMSLGGPADVPELHDAIKAAVNKNILVVCSMAAPHVSGALALIKDFANRQFERKLSEPELYAQLIRRTVPLGNSPKLEGNGLVYLTVPDHLAGIFDQELKSTVLNAI

SEQ ID NO: 14 - protein, 41 aa, B. anthracis Sterne
MSNNNYSNGLNPDESLSASAFDPNLVGPTLPPIPPFTLPTG